(19) United States
(12) Patent Application Publication
(10) Pub. No.: US 2026/0325510 A1
(43) Pub. Date: Oct. 22, 2026

(54) COMBINATIONS COMPRISING AN ATR INHIBITOR AND A PARP INHIBITOR AND METHODS OF USE THEREOF

(71) Applicants: Jazz Pharmaceuticals Therapeutics, Inc., Palo Alto, CA (US); The Governing Council of the University of Toronto, Toronto (CA)

(72) Inventors: Leon WAN, Vancouver (CA); Shyam GARG, Richmond (CA); Robert HAUPTSCHEIN, Ewing, NJ (US); Philippe LEGROS, Burnaby (CA); Paul TARDI, Surrey (CA); Lucy WANG, Ontario (CA); Christine ALLEN, Ontario (CA)

(21) Appl. No.: 19/485,955

(22) PCT Filed: Jul. 12, 2024
(86) PCT No.: PCT/US2024/037857

§ 371 Date: Nov. 19, 2025

Related U.S. Application Data
(60) Provisional application No. 63/513,744, filed on Jul. 14, 2023.

(51) Int. Cl. A61K 31/4965; A61K 9/127; A61K 31/454; A61K 31/4745; A61K 31/497; A61K 33/243; A61P 35/00

(52) U.S. Cl. CPC A61K 31/4965; A61K 9/127; A61K 31/454; A61K 31/4745; A61K 31/497; A61K 33/243; A61P 35/00

(57) ABSTRACT

The present disclosure relates to combinations comprising an ATR inhibitor and a PARP inhibitor and optionally further comprising an additional pharmaceutically active agent. Also provided are certain methods of treatment, e.g., a method for treating cancer such as ovarian cancer, with a combination comprising an effective amount of an ATR inhibitor and an effective amount of a PARP inhibitor and optionally further comprising an additional pharmaceutically active agent. In particular, a combination comprising a liposomal composition of an ATR inhibitor and a liposomal composition of a PARP inhibitor is disclosed herein.

### CROSS REFERENCE TO RELATED APPLICATIONS

This application claims the benefit of U.S. Provisional Application No. 63/513,744, filed Jul. 14, 2023, the disclosure of which is incorporated by reference herein in its entirety.

### FIELD OF THE INVENTION

The present disclosure generally relates to combinations comprising an ATR inhibitor and a PARP inhibitor and optionally an additional pharmaceutically active agent. Also provided are certain methods of treatment, e.g., a method for treating cancer such as ovarian cancer, with a combination comprising an effective amount of an ATR inhibitor and an effective amount of a PARP inhibitor and optionally an additional pharmaceutically active agent. In particular, a combination of a liposomal composition of an ATR inhibitor and a liposomal composition of a PARP inhibitor and optionally an additional pharmaceutically active agent is disclosed herein.

### BACKGROUND OF THE INVENTION

The ataxia-telangiectasia and Rad3-related (ATR) kinase is a serine/threonine protein kinase believed to be involved in the cellular DNA damage repair processes and cell cycle signaling. ATR kinase acts with ATM (“ataxia telangiectasia mutated”) kinase and other proteins to regulate a cell's response to DNA damage, commonly referred to as the DNA Damage Response (“DDR”). The DDR is believed to stimulate DNA repair, promote survival and stalls cell cycle progression by activating cell cycle checkpoints, which provide time for repair. Without the DDR, cells are much more sensitive to DNA damage and readily die from DNA lesions induced by endogenous cellular processes such as DNA replication or exogenous DNA damaging agents commonly used in cancer therapy.

The disruption of ATR function (e.g., by gene deletion) has been shown to promote cancer cell death both in the absence and presence of DNA damaging agents. Mutations of ATR have been linked to cancers of the stomach and endometrium, and lead to increased sensitivity to ionizing radiation and abolished cell cycle checkpoints. ATR is essential for the viability of somatic cells, and deletion of ATR has been shown to result in loss of damage checkpoint responses and cell death. See Cortez et al., Science 294:1713-1716 (2001). ATR is also essential for the stability of fragile sites, and low ATR expression in Seckel syndrome patients results in increased chromosomal breakage following replication stress. See Casper et al., Am. J. Hum. Genet 75:654-660 (2004). The replication protein A (RPA) complex recruits ATR, and its interacting protein ATRIP, to sites of DNA damage, and ATR itself mediates the activation of the CHK1 signaling cascade. See Zou et al., Science 300:1542-1548 (2003). ATR, like its related checkpoint kinase ATM, phosphorylates RAD17 early in a cascade that is critical to for checkpoint signaling in DNA-damaged cells. See Bao et al., Nature 411:969-974 (2001). It is believed that ATR is particularly essential in the early mammalian embryo, to sense incomplete DNA replication and prevent mitotic catastrophe.

Clinical success with the use of poly(ADP-ribose) polymerase (PARP) inhibitors (PARPi) for ovarian cancer has opened the medical landscape to the therapeutic potential of molecular therapy. While novel agents such as WEE1 and ATR inhibitors (ATRi) are gaining traction in clinical trials, dose-limiting toxicities such as anemia has impeded or prevented further development. See Roulston et al., Mol. Cancer Ther. 21 (2022) 245-256; Fang et al., Cancer Cell 35 (2019) 851-867.e7.

Thus, there is a need for effectively delivering the combination of an ATRi and a PARPi to treat cancer such that it eliminates dose-limiting toxicities.

U.S. Pat. Nos. 10,570,119, 11,028,076, and 11,787,781 disclose ATR inhibitors, each of the disclosures is hereby incorporated by reference in its entirety.

### SUMMARY OF THE INVENTION

The present disclosure provides combinations comprising an ATR inhibitor and a PARPi.

The present disclosure also provides combinations comprising a compound of formula (I) or a pharmaceutically acceptable salt, tautomer, or stereoisomer thereof, and a PARPi.

The present disclosure also provides combinations comprising an anti-cancer agent and a liposomal composition comprising a PARPi.

Each of the combination is a “combination of the invention”.

The present disclosure also provides methods for treating cancer, comprising administering to a subject in need thereof an effective amount of the combination of the invention (each method being a “method of the invention”).

### BRIEF DESCRIPTION OF THE DRAWINGS

FIG. 1A is a heat map showing combination index (CI) of various niraparib (“NIRB”) and ATRi (Compounds A, B, and C, and VE822) combinations at 0.75 fraction affected. FIG. 1B is a heat map showing CI of various NIRB and ATRi (Compounds A, B, and C, and VE822) combinations at 0.9 fraction affected.

FIG. 2A shows loading capacity of niraparib at various drug:lipid molar ratio. FIG. 2B shows encapsulation efficiency for niraparib at various drug:lipid molar ratio. FIG. 2C shows in vitro release of niraparib from liposomal formulation at 0.2 drug:lipid ratio. FIG. 2D shows in vitro release of niraparib from liposomal formulation at 0.4 drug:lipid ratio. FIG. 2E shows in vitro release of niraparib from liposomal formulation at 0.6 drug:lipid ratio. FIG. 2F shows in vitro release of niraparib from liposomal formulation at 0.8 drug:lipid ratio.

FIG. 3A show systemic toxicity in drug administered mice expressed as weight change over a 2-4 weeks study duration. FIG. 3B show systemic toxicity in drug administered mice expressed as survival over a 2-4 weeks study duration. FIGS. 3C-3N show results of CBC analysis after free niraparib monotherapy, liposomal niraparib monotherapy, and free or liposomal combination therapy with niraparib and Compound A or Compound C, over a 2-4 weeks study duration.

FIGS. 3C-3F show results of complete blood count (CBC) analysis after free niraparib monotherapy and liposomal niraparib monotherapy over a 2-4 week study duration: lymphocytes (FIG. 3C), neutrophils (FIG. 3D), monocytes (FIG. 3E), red blood cell counts (FIG. 3F). FIGS. 3G-3N show results of complete blood count (CBC) analysis after free or liposomal combination therapy with niraparib and an ATRi (Compound A, Compound C, or VE822) over a 2-4 week study duration: lymphocytes (FIGS. 3G and 3H), neutrophils (FIGS. 3I and 3J), monocytes (FIGS. 3K and 3L), red blood cell counts (FIGS. 3M and 3N). FIGS. 3O-3R shows fold change in CBC parameters when compared to relative controls.

FIGS. 4A-4D show concentration of niraparib and ATRi (Compounds A and C) over time. Half lives (FIGS. 5A-5B) and cumulative AUCs (FIGS. 5C-5D) were then calculated using the model of best fit (i.e., a 3-compartment model for free drug and a 1-compartment model for liposomal drug).

FIG. 4A shows blood concentration of niraparib and ATRi (Compound A) over time after administration of free niraparib and free ATRi. FIG. 4B shows blood concentration of niraparib and ATRi (Compound A) over time after administration of liposomal niraparib and liposomal ATRi.

FIG. 4C shows blood concentration of niraparib and ATRi (Compound C) over time after administration of free niraparib and free ATRi. FIG. 4D shows blood concentration of niraparib and ATRi (Compound C) over time after administration of liposomal niraparib and liposomal ATRi.

FIG. 5A show half-lives of niraparib and ATRi (Compound A) in free or liposomal formulations. FIG. 5B show half-lives of niraparib and ATRi (Compound C) in free or liposomal formulations. FIG. 5C shows calculated cumulative AUCs of niraparib and ATRi (Compound A) administered as various free or liposomal formulations. FIG. 5D shows calculated cumulative AUCs of niraparib and ATRi (Compound C) administered as various free or liposomal formulations.

FIG. 6 shows change in mean tumor volume when free niraparib is administered alone or in combination with Compound A in a liposomal formulation (triple negative breast cancer MDA-MB-436 model).

FIG. 7A shows erythroid toxicity of the combination of niraparib and liposomal Compound A vs free drug. Bracketed %'s represent percent improvement of Erythroid toxicity by the liposomal agent vs free drug. FIG. 7B shows results of hematology tolerability study for free niraparib alone or in combination with Compound A and Compound C in a liposomal formulation.

FIG. 8A shows the change in OVCAR8 tumor volume (mm³) after treatment with a triple combination of (i) liposomal niraparib (in Formulation B) administered at 60 mg/kg, liposomal Compound A (in Formulation B) administered at 60 mg/kg, and free cisplatin administered at 1 mg/kg; (ii) liposomal niraparib (in Formulation A) administered at 60 mg/kg, liposomal Compound A (in Formulation A) administered at 60 mg/kg, and free cisplatin administered at 1 mg/kg (iii) liposomal niraparib (in Formulation B) administered at 80 mg/kg, liposomal Compound A (in Formulation B) administered at 80 mg/kg, and free CISPLATIN administered at 1 mg/kg; and (iv) liposomal niraparib (in Formulation A) administered at 80 mg/kg, liposomal Compound A (in Formulation A) administered at 80 mg/kg, and free cisplatin administered at 1 mg/kg using a OVCAR8 tumor model.

FIG. 8B shows the change in OVCAR8 tumor volume (mm³) after treatment with irinotecan at 50 mg/kg and liposomal niraparib (in Formulation B) administered at 80 mg/kg, liposomal Compound A (in Formulation B) administered at 80 mg/kg, and free irinotecan administered at 50 mg/kg.

FIG. 8C shows the change in OVCAR8 tumor volume (mm³) after treatment with (i) liposomal Compound A (in Formulation B) administered at 80 mg/kg, (ii) liposomal niraparib (in Formulation B) administered at 80 mg/kg, (iii) liposomal Compound A (in Formulation B) administered at 80 mg/kg and liposomal niraparib (in Formulation B) administered at 80 mg/kg; (iv) irinotecan administered at 50 mg/kg, (v) liposomal Compound A (in Formulation B) administered at 80 mg/kg and irinotecan 50 mg/kg; (vi) liposomal niraparib (in Formulation B) administered at 80 mg/kg and irinotecan administered at 50 mg/kg and (vii) liposomal niraparib (in Formulation B) administered at 80 mg/kg, and liposomal Compound A (in Formulation B) administered at 80 mg/kg and irinotecan administered at 50 mg/kg.

FIG. 9 shows tumor reduction with the niraparib and Compound A combination as either free or liposomal form.

### DETAILED DESCRIPTION

All publications, patents and patent applications, including any drawings and appendices therein are incorporated by reference in their entirety for all purposes to the same extent as if each individual publication, patent or patent application, drawing, or appendix was specifically and individually indicated to be incorporated by reference in its entirety for all purposes.

### Definitions

While the following terms are believed to be well understood by one of ordinary skill in the art, the following definitions are set forth to facilitate explanation of the presently disclosed subject matter.

Throughout the present specification, the terms “about” and/or “approximately” may be used in conjunction with numerical values and/or ranges. The term “about” is understood to mean those values near to a recited value. Furthermore, the phrases “less than about [a value]” or “greater than about [a value]” should be understood in view of the definition of the term “about” provided herein. The terms “about” and “approximately” may be used interchangeably.

Throughout the present specification, numerical ranges are provided for certain quantities. It is to be understood that these ranges comprise all subranges therein. Thus, the range “from 50 to 80” includes all possible ranges therein (e.g., 51-79, 52-78, 53-77, 54-76, 55-75, 60-70, etc.). Furthermore, all values within a given range may be an endpoint for the range encompassed thereby (e.g., the range 50-80 includes the ranges with endpoints such as 55-80, 50-75, etc.).

The term “a” or “an” refers to one or more of that entity; for example, “an ATR inhibitor” refers to one or more ATR inhibitor or at least one ATR inhibitor. As such, the terms “a” (or “an”), “one or more” and “at least one” are used interchangeably herein. In addition, reference to “an inhibitor” by the indefinite article “a” or “an” does not exclude the possibility that more than one of the inhibitors is present, unless the context clearly requires that there is one and only one of the inhibitors.

As used herein, the verb “comprise” as is used in this description and in the claims and its conjugations are used in its non-limiting sense to mean that items following the word are included, but items not specifically mentioned are not excluded. The present invention may suitably “comprise”, “consist of”, or “consist essentially of”, the steps, elements, and/or reagents described in the claims.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

The term “pharmaceutically acceptable salts” includes both acid and base addition salts. Pharmaceutically acceptable salts include those obtained by reacting the active compound functioning as a base, with an inorganic or organic acid to form a salt, for example, salts of hydrochloric acid, sulfuric acid, phosphoric acid, methanesulfonic acid, camphorsulfonic acid, oxalic acid, maleic acid, succinic acid, citric acid, formic acid, hydrobromic acid, benzoic acid, tartaric acid, fumaric acid, salicylic acid, mandelic acid, carbonic acid, etc. Those skilled in the art will further recognize that acid addition salts may be prepared by reaction of the compounds with the appropriate inorganic or organic acid via any of a number of known methods.

The term “treating” means one or more of relieving, alleviating, delaying, reducing, improving, or managing at least one symptom of a condition in a subject. The term “treating” may also mean one or more of arresting, delaying the onset (i.e., the period prior to clinical manifestation of the condition) or reducing the risk of developing or worsening a condition.

An “effective amount” means the amount of a formulation according to the invention that, when administered to a patient for treating a state, disorder or condition is sufficient to effect such treatment. The “effective amount” will vary depending on the active ingredient, the state, disorder, or condition to be treated and its severity, and the age, weight, physical condition and responsiveness of the mammal to be treated.

The term “therapeutically effective” applied to dose or amount refers to that quantity of a compound or pharmaceutical formulation that is sufficient to result in a desired clinical benefit after administration to a patient in need thereof.

The terms “pharmaceutical combination,” “therapeutic combination” or “combination” as used herein, refers to a single composition or dosage form comprising at least two therapeutically active agents (e.g., ATRi and PARPi), or separate composition or dosage forms comprising at least two therapeutically active agents together or separately for use in a combination therapy. For example, one therapeutically active agent may be formulated into one composition or dosage form and the other therapeutically active agent may be formulated into a single or different compositions or dosage forms; these can be given together or separately. For example, one therapeutically active agent (e.g., ATRi) may be formulated into a first liposomal formulation whereas the second therapeutically active agent (e.g., PARPi) may be formulated into a second liposomal formulation. In some embodiments, the combination includes one or more kits.

The term “combination therapy” as used herein refers to a first therapy that comprises an ATRi in conjunction with a second therapy that comprises a PARPi and optionally a third therapy that comprises an additional pharmaceutically active agent, such as an anti-cancer agent that is useful for treating, ameliorating, and/or delaying the disease or condition.

Administration in “conjunction with” another therapeutically active agent includes administration in the same or different composition(s) and/or combinations, either sequentially, simultaneously, or continuously, through the same or different routes. In some embodiments, two or more therapeutically active agents that are used in combination therapy are on the same or different dosing schedule which can start at the same time or separately. For example, in one embodiment of the method of the invention, ATRi can be administered once a week and PARPi can be administered once every three days.

The term “liposome” as used herein means vesicles comprised of one or more concentrically ordered lipid bilayers encapsulating an aqueous phase. Formation of such vesicles requires the presence of vesicle-forming lipids (also referred to as “liposome lipids” herein) which are amphipathic lipids capable of either forming or being incorporated into a bilayer structure. The latter term includes lipids that are capable of forming a bilayer by themselves or when in combination with another lipid or lipids. An amphipathic lipid is incorporated into a lipid bilayer by having its hydrophobic moiety in contact with the interior, hydrophobic region of the membrane bilayer and its polar head moiety oriented toward an outer, polar surface of the membrane. Hydrophilicity arises from the presence of functional groups such as hydroxyl, phosphato, carboxyl, sulfato, amino or sulfhydryl groups. Hydrophobicity results from the presence of a long chain of aliphatic hydrocarbon groups.

The term “non-liposomal,” “liposome-free,” “free drug (fd)” or “free” formulation or active ingredient as used herein refers to a therapeutically active ingredient/agent (such as ATRi or PARPi) that is not encapsulated in a liposome and is administered, in a formulation or solution that is not intended to control the release rate of the therapeutically active ingredient/agent.

As used herein, a “subject” can be a human, non-human primate, mammal, rat, mouse, cow, horse, pig, sheep, goat, dog, cat and the like. The subject can be suspected of having or at risk for having a cancer, including but not limited to colorectal cancer and melanoma.

“Mammal” includes humans and both domestic animals such as laboratory animals (e.g., mice, rats, monkeys, dogs, etc.) and household pets (e.g., cats, dogs, swine, cattle, sheep, goats, horses, rabbits), and non-domestic animals such as wildlife and the like.

All weight percentages (i.e., “% by weight” and “wt. %” and w/w) referenced herein, unless otherwise indicated, are measured relative to the total weight of the pharmaceutical composition.

“Alkyl” or “alkyl group” refers to a fully saturated, straight or branched hydrocarbon chain group, and which is attached to the rest of the molecule by a single bond. Alkyls comprising any number of carbon atoms, including but not limited to from 1 to 12 are included. An alkyl comprising up to 12 carbon atoms is a C₁-C₁₂ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₅ alkyl. A C₁-C₅ alkyl includes C₅ alkyls, C₄ alkyls, C₃ alkyls, C₂ alkyls and C₁ alkyl (i.e., methyl). A C₁-C₆ alkyl includes all moieties described above for C₁-C₅ alkyls but also includes C₆ alkyls. A C₁-C₁₀ alkyl includes all moieties described above for C₁-C₅ alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples of C₁-C₁₂ alkyl include methyl, ethyl, n-propyl, i-propyl, sec-propyl, n-butyl, i-butyl, sec-butyl, t-butyl, n-pentyl, t-amyl, n-hexyl, n-heptyl, n-octyl, n-Nonyl, n-decyl, n-undecyl, and n-dodecyl. Unless stated otherwise specifically in the specification, an alkyl group can be optionally substituted.

“Alkylene” refers to a fully saturated, straight or branched hydrocarbon chain group, and which is attached to the rest of the molecule by two single bonds (divalent) —i.e., an alkyl group as defined above that has one additional point of attachment. Unless stated otherwise specifically in the specification, an alkylene group can be optionally substituted.

The term “substituted” used herein means any of the above groups (i.e., alkyl, alkylene, alkenyl, alkenylene, alkynyl, alkynylene, alkoxy, alkylamino, alkylcarbonyl, thioalkyl, aryl, aralkyl, carbocyclyl, cycloalkyl, cycloalkenyl, cycloalkynyl, cycloalkylalkyl, haloalkyl, heterocyclyl, N-heterocyclyl, heterocyclylalkyl, heteroaryl, N-heteroaryl and/or heteroarylalkyl) wherein at least one hydrogen atom is replaced by a bond to a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. “Substituted also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl heteroarylalkyl. “Substituted” further means any of the above groups in which one or more hydrogen atoms are replaced by a bond to an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group. In addition, each of the foregoing groups can also be optionally substituted with one or more of the above groups.

### Compounds Useful in the Combinations and the Methods of the Invention

The present disclosure relates to combinations comprising a compound of formula (I) or a pharmaceutically acceptable salt, tautomer, or stereoisomer thereof, and a PARPi. The present disclosure also relates to the therapeutic use of the combinations.

### ATR Inhibitors

The combination of the invention comprises a compound of formula (I):

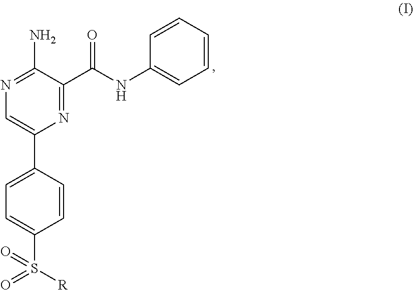

or a pharmaceutically acceptable salt thereof, wherein R is a moiety comprising an amino group with a pKₐ of greater than 7.0.

In some embodiments, the compound of formula (I) or a pharmaceutically acceptable salt thereof is an ATR inhibitor.

In some embodiments of the compound of formula (I), R is a moiety comprising an amino group with a pKₐ of greater than 8.0. In some embodiments, R is a moiety comprising an amino group with a pKₐ of greater than 8.5. In some embodiments, R is a moiety comprising an amino group with a pKₐ of greater than 9.0. In some embodiments, R is a moiety comprising an amino group with a pKₐ of greater than 9.5.

In some embodiments of the compound of formula (I), R is -alkylene-NR^{a}R^{b}, -(alkylene)-(heterocyclyl)-(alkyl), -(alkylene)-(heterocyclyl)-(alkylene)-NR^{a}R^{b}, -(alkylene)-(heterocyclyl)-NR^{a}R^{b}, or —NR^{a}R^{b}-(alkyl), wherein the heterocyclyl contains at least one nitrogen as a ring atom; and R^{a} and R^{b} are each independently H or alkyl. In some embodiments of the compound of formula (I), R is —C₁-C₄ alkylene-NR^{a}R^{b}, —(C₁-C₆ alkylene)-(heterocyclyl)-(C₁-C₆ alkyl), —(C₁-C₆ alkylene)-(heterocyclyl)-(C₁-C₆ alkylene)-NR^{a}R^{b}, —(C₁-C₆ alkylene)-(heterocyclyl)-NR^{a}R^{b}, or —NR^{a}R^{b}—(C₁-C₆ alkyl), wherein the heterocyclyl contains at least one nitrogen as a ring atom; and R^{a} and R^{b} are each independently H or —C₁-C₆ alkyl.

In some embodiments of the compound of formula (I), R is —C₁-C₄ alkylene-NR^{a}R^{b}, —(C₁-C₄ alkylene)-(heterocyclyl)-(C₁-C₄ alkyl), —(C₁-C₄ alkylene)-(heterocyclyl)-(C₁-C₄ alkylene)-NR^{a}R^{b}, —(C₁-C₄ alkylene)-(heterocyclyl)-NR^{a}R^{b}, or —NR^{a}R^{b}—(C₁-C₄ alkyl), wherein the heterocyclyl contains at least one nitrogen as a ring atom; and R^{a} and R^{b} are each independently H or —C₁-C₄ alkyl. In some embodiments, the heterocyclyl is a divalent heterocycle containing one nitrogen as a ring atom and optionally one additional heteroatom selected from N, S, or O. In some embodiments, the heterocyclyl is a divalent heterocycle containing one nitrogen as a ring atom. In some embodiments, the heterocyclyl is a divalent heterocycle containing two nitrogen atoms as ring atoms. In some embodiments, the heterocyclyl is a divalent 5- to 7-membered heterocycle containing at least one nitrogen as a ring atom. In some embodiments, the heterocyclyl is a divalent 6-membered heterocycle containing at least one nitrogen as a ring atom. In some embodiments, the heterocyclyl is a divalent 6-membered heterocycle containing at least one nitrogen as a ring atom and optionally one additional heteroatom selected from N, S, or O. In some embodiments, the heterocyclyl is a divalent 6-membered heterocycle containing one nitrogen as a ring atom. In some embodiments, the heterocyclyl is a divalent 6-membered heterocycle containing two nitrogen atoms as ring atoms. In some embodiments, the heterocyclyl is a divalent 5- to 7-membered saturated heterocycle containing at least one nitrogen as a ring atom. In some embodiments, the heterocyclyl is a divalent 6-membered saturated heterocycle containing at least one nitrogen as a ring atom. In some embodiments, the heterocyclyl is a divalent 6-membered saturated heterocycle containing at least one nitrogen as a ring atom and optionally one additional heteroatom selected from N, S, or O. In some embodiments, the heterocyclyl is a divalent 6-membered saturated heterocycle containing one nitrogen as a ring atom. In some embodiments, the heterocyclyl is a divalent 6-membered saturated heterocycle containing two nitrogen atoms as ring atoms.

In some embodiments of the compound of formula (I), R is —(C₁-C₄ alkylene)-(heterocyclyl)-(C₁-C₄ alkyl), —(C₁-C₄ alkylene)-(heterocyclyl)-(C₁-C₄ alkylene)-NR^{a}R^{b}, or —(C₁-C₄ alkylene)-(heterocyclyl)-NR^{a}R^{b}, wherein the heterocyclyl is a piperidine or a piperazine.

In some embodiments of the compound of formula (I), R is:
a)

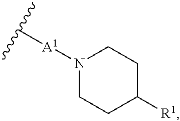

wherein A¹ is absent or C₁-C₄ alkylene, and R¹ is —(C₁-C₄ alkyl)-NR^{a}R^{b}, wherein R^{a} and R^{b} are each independently H or —C₁-C₄ alkyl;
b) —N(R^{a})(C₁-C₄ alkyl)-NR^{a}R^{b}, wherein R^{a} and R^{b} are each independently H or —C₁-C₄ alkyl;
c) —(C₁-C₈ alkyl)-NR^{a}R^{b}, wherein R^{a} and R^{b} are each independently H or C₁-C₄ alkyl; or
d)

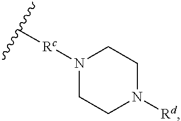

wherein R^{c} is —C₁-C₄ alkylene and R^{d} is —C₁-C₄ alkyl.

In some embodiments of the compound of formula (I), R is

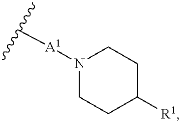

wherein A¹ is absent or alkylene, and R¹ is -(alkylene)-NR^{a}R^{b}, wherein R^{a} and R^{b} are each independently H or alkyl. In some embodiments, R is

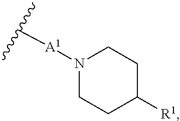

wherein A¹ is absent or C₁-C₆ alkylene, and R¹ is —(C₁-C₆ alkylene)-NR^{a}R^{b}, wherein R^{a} and R^{b} are each independently H or —C₁-C₆ alkyl. In some embodiments, R is

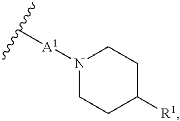

wherein A¹ is absent or C₁-C₄ alkylene, and R¹ is —(C₁-C₄ alkyl)-NR^{a}R^{b}, wherein R^{a} and R^{b} are each independently H or —C₁-C₄ alkyl. In some embodiments, A¹ is C₁-C₄ alkylene. In some embodiments, A¹ is a straight or branched C₁-C₄ alkylene. In some embodiments, A¹ is —CH₂—, —CH₂CH₂—, or —CH₂CH₂CH₂—. In some embodiments, A¹ is —CH₂CH₂—.

In some embodiments of the compound of formula (I), R is —N(R^{a})(alkylene)-NR^{a}R^{b}, wherein R^{a} and R^{b} are each independently H or alkyl. In some embodiments, R is —N(R^{a})(C₁-C₆ alkylene)-NR^{a}R^{b}, wherein R^{a} and R^{b} are each independently H or —C₁-C₆ alkyl. In some embodiments, R is —N(R^{a})(C₁-C₄ alkylene)-NR^{a}R^{b}, wherein R^{a} and R^{b} are each independently H or —C₁-C₄ alkyl. In some embodiments, R is —N(R^{a})(C₁-C₄ alkylene)-NR^{a}R^{b}, wherein C₁-C₄ alkylene is straight or branched; and R^{a} and R^{b} are each independently H or —C₁-C₄ alkyl. In some embodiments, R is —N(H)(C₁-C₄ alkylene)-NR^{a}R^{b}, wherein R^{a} and R^{b} are each independently H or —C₁-C₄ alkyl. In some embodiments, R is —NHCH₂—NR^{a}R^{b}, —NHCH₂CH₂—NR^{a}R^{b}, or —NHCH₂CH₂CH₂—NR^{a}R^{b}, or wherein R^{a} and R^{b} are each independently H or —C₁-C₄ alkyl. In some embodiments, R is —NHCH₂—NR^{a}R^{b}, —NHCH₂CH₂—NR^{a}R^{b}, or —NHCH₂CH₂CH₂—NR^{a}R^{b}, or wherein R^{a} and R^{b} are each independently-C₁-C₄ alkyl. In some embodiments, R is —NHCH₂—NR^{a}R^{b}, —NHCH₂CH₂—NR^{a}R^{b}, or —NHCH₂CH₂CH₂—NR^{a}R^{b}, or wherein R^{a} and R^{b} are each independently methyl, ethyl, or propyl. In some embodiments, R is —NHCH₂CH₂—NR^{a}R^{b}, wherein R^{a} and R^{b} are each methyl.

In some embodiments of the compound of formula (I), R is -(alkylene)-NR^{a}R^{b}, wherein R^{a} and R^{b} are each independently H or alkyl. In some embodiments, R is —(C₁-C₁₀ alkylene)-NR^{a}R^{b}, wherein R^{a} and R^{b} are each independently H or C₁-C₆ alkyl. In some embodiments, R is —(C₁-C₈ alkylene)-NR^{a}R^{b}, wherein R^{a} and R^{b} are each independently H or C₁-C₄ alkyl. In some embodiments, R is —(C₁-C₈ alkylene)-NR^{a}R^{b} wherein C₁-C₈ alkylene is straight or branched; and R^{a} and R^{b} are each independently H or C₁-C₄ alkyl. In some embodiments, R is —(C₁-C₄ alkylene)-NR^{a}R^{b}, wherein R^{a} and R^{b} are each independently H or C₁-C₄ alkyl. In some embodiments, R is —(C₁-C₄ alkylene)-NR^{a}R^{b} wherein C₁-C₄ alkylene is straight or branched; and R^{a} and R^{b} are each independently H or C₁-C₄ alkyl. In some embodiments, R is —(C₁-C₄ alkylene)-NR^{a}R^{b}, wherein R^{a} and R^{b} are each independently H or C₁-C₄ alkyl. In some embodiments, R is —CH₂NR^{a}R^{b}, —CH₂CH₂NR^{a}R^{b}, —CH₂CH₂CH₂NR^{a}R^{b}, or —CH(CH₃) CH₂NR^{a}R^{b}, wherein R^{a} and R^{b} are each independently H or C₁-C₄ alkyl. In some embodiments, R is —CH₂NR^{a}R^{b}, —CH₂CH₂NR^{a}R^{b}, —CH₂CH₂CH₂NR^{a}R^{b}, or —CH(CH₃) CH₂NR^{a}R^{b}, wherein R^{a} and R^{b} are each independently C₁-C₄ alkyl. In some embodiments, R is —CH₂NR^{a}R^{b}, —CH₂CH₂NR^{a}R^{b}, —CH₂CH₂CH₂NR^{a}R^{b}, or —CH(CH₃) CH₂NR^{a}R^{b}, wherein R^{a} and R^{b} are each independently methyl, ethyl, or propyl. In some embodiments, R is —CH₂NR^{a}R^{b}, —CH₂CH₂NR^{a}R^{b}, —CH₂CH₂CH₂NR^{a}R^{b}, or —CH(CH₃) CH₂NR^{a}R^{b}, wherein R^{a} and R^{b} are each independently methyl or ethyl. In some embodiments, R is —CH(CH₃) CH₂NR^{a}R^{b}, wherein R^{a} and R^{b} are each independently methyl, ethyl, or propyl. In some embodiments, R is —CH₂NR^{a}R^{b}, —CH₂CH₂NR^{a}R^{b}, —CH₂CH₂CH₂NR^{a}R^{b}, or —CH(CH₃) CH₂NR^{a}R^{b}, wherein R^{a} and R^{b} are each methyl. In some embodiments, R is —CH₂NR^{a}R^{b}, —CH₂CH₂NR^{a}R^{b}, —CH₂CH₂CH₂NR^{a}R^{b}, or —CH(CH₃) CH₂NR^{a}R^{b}, wherein R^{a} and R^{b} are each independently methyl, ethyl, or propyl. In some embodiments, R is —CH₂CH₂NR^{a}R^{b}, wherein R^{a} and R^{b} are each ethyl.

In some embodiments of the compound of formula (I), R is

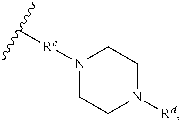

wherein R^{c} is alkylene and R^{d} is alkyl. In some embodiments, R is

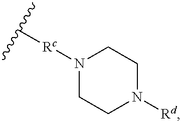

wherein R^{c} is —C₁-C₆ alkylene and R^{d} is —C₁-C₆ alkyl. In some embodiments, R is

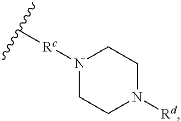

wherein R^{c} is —C₁-C₄ alkylene and R^{d} is —C₁-C₄ alkyl. In some embodiments, R is

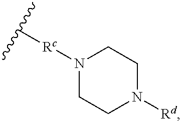

wherein R^{c} is —C₁-C₄ alkylene wherein-C₁-C₄ alkylene is straight or branched, and R^{d} is —C₁-C₄ alkyl. In some embodiments, R is

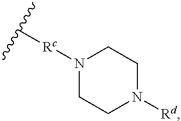

wherein Re is —CH₂—, —CH₂CH₂—, or —CH₂CH₂CH₂— and R^{d} is methyl, ethyl, or propyl. In some embodiments, R is

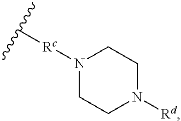

wherein R^{c} is —CH₂CH₂— and R^{d} is methyl.

In some embodiments, the compound of formula (I) is selected from Table A, or a pharmaceutically acceptable salt thereof. In some embodiments, the compound of formula (I) is Compound 1, Compound 2, Compound 3, Compound 4, Compound 5, or Compound 6, or a pharmaceutically acceptable salt thereof.

TABLE A
Compounds of Formula (I)

| | |
|---|---|
| 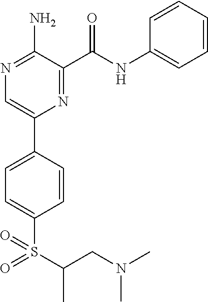 [SMILES: CC(CN(C)C)S(=O)(=O)c1ccc(-c2cnc(N)c(C(=O)Nc3ccccc3)n2)cc1] | Compound 1 |
| 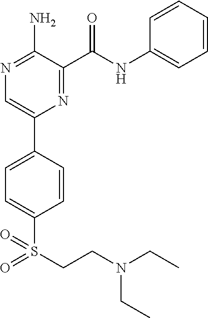 [SMILES: CCN(CC)CCS(=O)(=O)c1ccc(-c2cnc(N)c(C(=O)Nc3ccccc3)n2)cc1] | Compound 2 |
| 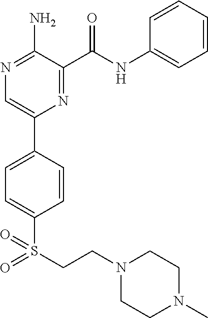 [SMILES: CN1CCN(CCS(=O)(=O)c2ccc(-c3cnc(N)c(C(=O)Nc4ccccc4)n3)cc2)CC1] | Compound 3 |
| 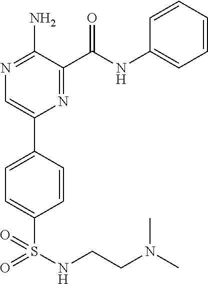 [SMILES: CN(C)CCNS(=O)(=O)c1ccc(-c2cnc(N)c(C(=O)Nc3ccccc3)n2)cc1] | Compound 4 |
| 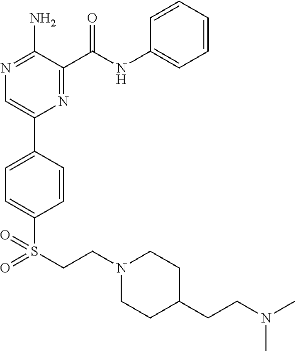 [SMILES: CN(C)CCC1CCN(CCS(=O)(=O)c2ccc(-c3cnc(N)c(C(=O)Nc4ccccc4)n3)cc2)CC1] | Compound 5 |
| 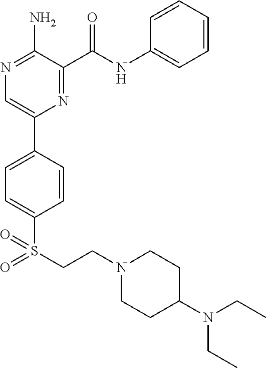 [SMILES: CCN(CC)C1CCN(CCS(=O)(=O)c2ccc(-c3cnc(N)c(C(=O)Nc4ccccc4)n3)cc2)CC1] | Compound 6 |

In embodiments, the ATR inhibitor is berzosertib.

### PARP Inhibitors

The combination of the invention comprises a PARP inhibitor.

In some embodiments, the PARP inhibitor is olaparib, rucaparib, niraparib, talazoparib, veliparib, fluzoparib, pamiparib, fluazolepali, amelparib, simmiparib, mefuparib, iniparib, stenoparib, senaparib, atamparib, venadaparib, or nesuparib, or a pharmaceutically acceptable salt thereof. In some embodiments, the PARP inhibitor is niraparib.

In some embodiments, the PARP inhibitor is talazoparib, niraparib, rucaparib, olaparib, fluzoparib, pamiparib, fluazolepali, veliparib, amelparib, CK-102, CEP 9722 (4,5,6,7-Tetrahydro-11-methoxy-2-((4-methyl-1-piperazinyl)methyl)-1H-cyclopenta(a)pyrrolo(3,4-C)carbazole-1,3(2H)-dione), CEP-8983 (11-methoxy-4,5,6,7-tetrahydro-1H-cyclopenta[a]pyrrolo[3,4-c]carbazole-1,3(2H)-dione), stenoparib, simmiparib, SC-10914, senaparib, ABT-767, MP-124, atamparib, venadaparib, nesuparib, HWH-340, ZYTP-1, NT-125, AST-6828, OX401, HC—X014, CBX-11, OC-301, TSL-1502, TSL-1502M, mefuparib, R-554, AZ-6102 (2-(4-(6-((3R,5S)-3,5-dimethylpiperazin-1-yl)-4-methylpyridin-3-yl)phenyl)-7-methyl-3,7-dihydro-4H-pyrrolo[2,3-d]pyrimidin-4-one), BPI-7000, SRX-3128, JNJ-928, AZD-2461 (4-(4-fluoro-3-(4-methoxypiperidine-1-carbonyl)benzyl) phthalazin-1 (2H)-one), iniparib, ONO-2231, INO-1001 (N-(3-Morpholinopropyl)-5-oxo-6,11-dihydro-5H-indeno[1,2-c]isoquinoline-9-sulfonamide), INO-1003, E-7016, LT-626, JPI-283, MK-2512, R-503, NMS-P914A, HYDAMTIQ, KR-33889, S-111, ANG-2864, PD-141703, PD-141076 (6,11-dihydro[1]benzothiopyrano[4,3-b]indole), PD-128763 (5-methyl-3,4-dihydroisoquinolin-1 (2H)-one), BSI-401, A-620223 (2-(1-Propylpiperidin-4-yl)-1H-benzo[d]imidazole-7-carboxamide), AAI-028, DR-2313 (7,8-dihydro-2-methyl-1H-thiopyrano[4,3-d]pyrimidin-4(5H)-one), BGP-15 (N-(2-hydroxy-3-(piperidin-1-yl)propoxy)nicotinimidamide), LT-673 (5-Fluoro-8-(4-fluorophenyl)-9-(1-methyl-1H-1,2,4-triazol-5-yl)-8,9-dihydro-2H-pyrido[4,3,2-de]phthalazin-3 (7H)-one), NMS-P118 (2-[1-(4,4-Difluorocyclohexyl)-piperidin-4-yl]-6-fluoro-3-oxo-2,3-dihydro-1H-isoindole-4-carboxamide), XAV939 (3,5,7,8-Tetrahydro-2-[4-(trifluoromethyl)phenyl]-4H-thiopyrano[4,3-d]pyrimidin-4-one), or a pharmaceutically acceptable salt thereof.

### Other Pharmaceutically Active Agents

In embodiments, the combination of the present disclosure comprises an additional pharmaceutically active agent. In embodiments, the additional pharmaceutically active agent is one or more anti-cancer agents.

In embodiments, the additional pharmaceutically active agent is a camptothecin derivative such as topotecan or irinotecan or a pharmaceutically acceptable salt thereof.

In embodiments, the anti-cancer agent is a derivative such as topotecan or irinotecan. In embodiments, the anti-cancer agent is an alkylating agent, for example platinum coordination complexes such as cisplatin, carboplatin and oxaliplatin.

In embodiments, the anti-cancer agent is irinotecan:

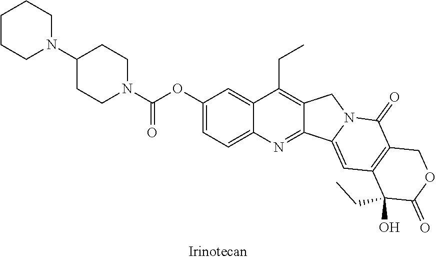 [SMILES: CCc1c2c(nc3ccc(OC(=O)N4CCC(N5CCCCC5)CC4)cc13)-c1cc3c(c(=O)n1C2)COC(=O)[C@]3(O)CC]

In some embodiments, the additional pharmaceutically active agent is irinotecan or a pharmaceutically acceptable salt thereof.

In embodiments, the anti-cancer agent is cisplatin.

In embodiments, the anti-cancer agent is a DNA damaging agent. In embodiments, the DNA damaging agent is carboplatin, cisplatin, cyclophosphamide, doxorubicin, daunorubicin, epirubicin, mitomycin C, or mitoxantrone.

In embodiments, the anti-cancer agent is a DNA repair inhibitor. In embodiments, the DNA repair inhibitor is 5-fluorouracil (5-FU), fluorodeoxyuridine (FUDR), gemcitabine or methotrexate. In embodiments, the anti-cancer agent is gemcitabine.

In embodiments, the anti-cancer agent is a topoisomerase I inhibitor. In embodiments, the topoisomerase I inhibitor is camptothecin, irinotecan or topotecan. In embodiments, the anti-cancer agent is irinotecan or topotecan.

In embodiments, the anti-cancer agent is a S/G2 or G2/M checkpoint inhibitor. In embodiments, the S/G2 or G2/M checkpoint inhibitor is bleomycin, docetaxel, doxorubicin, etoposide, paclitaxel, vinblastine, vincristine, vindesine or vinorelbine.

In embodiments, the anti-cancer agent is a G1/early-S checkpoint inhibitor. In embodiments, the anti-cancer agent is a G2/M checkpoint inhibitor.

In embodiments, the anti-cancer agent is a receptor tyrosine kinase inhibitor. In embodiments, the receptor tyrosine kinase inhibitor is genistein, trastuzumab, ZD1839.

In embodiments, the anti-cancer agent is a cytotoxic agent. In embodiments, the anti-cancer agent is an apoptosis-inducing agent. In embodiments, the anti-cancer agent is a cell cycle control inhibitor.

### Pharmaceutical Compositions and Combinations

The present disclosure also relates the combinations of the invention, wherein the ATR inhibitor is provided in a pharmaceutical composition. In some embodiments, the pharmaceutical composition comprises an ATR inhibitor and a pharmaceutically acceptable carrier or excipient.

The present disclosure also relates the combinations of the invention, wherein the compounds of formula (I) or a pharmaceutically acceptable salt thereof is provided in a pharmaceutical composition. In some embodiments, the pharmaceutical composition comprises a compound of formula (I) or a pharmaceutically acceptable salt thereof and a pharmaceutically acceptable carrier or excipient.

The present disclosure also relates the combinations of the invention, wherein the PARP inhibitor is provided in a pharmaceutical composition. In some embodiments, the pharmaceutical composition comprises a PARP inhibitor and a pharmaceutically acceptable carrier or excipient.

In some embodiments of the combinations of the invention, the combination comprises a pharmaceutical composition comprising an ATR inhibitor or a pharmaceutically acceptable salt thereof, and a pharmaceutical composition comprising a PARP inhibitor. In some embodiments of the combinations of the invention, the combination comprises a pharmaceutical composition comprising an ATR inhibitor or a pharmaceutically acceptable salt thereof and a pharmaceutically acceptable carrier or excipient, and a pharmaceutical composition comprising a PARP inhibitor and a pharmaceutically acceptable carrier or excipient.

In some embodiments of the combinations of the invention, the combination comprises a pharmaceutical composition comprising a compound of formula (I) or a pharmaceutically acceptable salt thereof, and a pharmaceutical composition comprising a PARP inhibitor. In some embodiments of the combinations of the invention, the combination comprises a pharmaceutical composition comprising a compound of formula (I) or a pharmaceutically acceptable salt thereof and a pharmaceutically acceptable carrier or excipient, and a pharmaceutical composition comprising a PARP inhibitor and a pharmaceutically acceptable carrier or excipient.

In some embodiments, the pharmaceutical composition comprising an ATR inhibitor is separate or different from the pharmaceutical composition comprising a PARP inhibitor. In some embodiments, the pharmaceutical composition comprising a compound of formula (I) or a pharmaceutically acceptable salt thereof is separate or different from the pharmaceutical composition comprising a PARP inhibitor.

In some embodiments, the pharmaceutical composition comprising an ATR inhibitor is the same as the pharmaceutical composition comprising a PARP inhibitor. In some embodiments, the pharmaceutical composition comprising an ATR inhibitor also comprises a PARP inhibitor. In some embodiments, the pharmaceutical composition comprising a compound of formula (I) or a pharmaceutically acceptable salt thereof is the same as the pharmaceutical composition comprising a PARP inhibitor. In some embodiments, the pharmaceutical composition comprising a compound of formula (I) or a pharmaceutically acceptable salt thereof also comprises a PARP inhibitor.

The present disclosure also relates the combinations of the invention, wherein the PARP inhibitor is provided in a liposomal composition.

The present disclosure also relates to the combinations of the invention comprising an anti-cancer agent and a liposomal composition comprising a PARPi. In some embodiment, the anti-cancer agent is irinotecan. In some embodiment, the PARPi is niraparib.

In some embodiments of any one of the pharmaceutical compositions as disclosed herein, the composition further comprises an additional therapeutic agent. The additional pharmaceutically active agent may be an anti-cancer agent.

In some embodiments, the pharmaceutical composition is a cream, gel, lotion, solution or liquid, suspension, solid, syrup, powder or granule.

In some embodiments, the pharmaceutical composition is formulated for oral administration, topical administration, parenteral administration (including intravenous, intraarterial, intraperitoneal, subcutaneous, intramuscular, intravascular or infusion); rectal administration, or by inhalation (including aerosol). In some embodiments, the pharmaceutical composition is formulated for administration intravenously or intraperitoneally by a bolus injection or infusion.

In some embodiments, the pharmaceutical composition comprising an ATR inhibitor is a liposomal composition. In some embodiments, the liposome composition comprises an ATR inhibitor, as the only pharmaceutically active agent.

In some embodiments, the pharmaceutical composition comprising a compound of formula (I) or a pharmaceutically acceptable salt thereof is a liposomal composition. In some embodiments, the liposome composition comprises a compound of formula (I), or a pharmaceutically acceptable salt thereof, as the only pharmaceutically active agent.

In some embodiments, the pharmaceutical composition comprising a PARP inhibitor is a liposomal composition. In some embodiments, the composition comprises a PARP inhibitor as the only pharmaceutically active agent.

Liposomal compositions can provide desirable pharmacokinetic properties for the compounds of an ATR inhibitor or the PARP inhibitor useful in the combinations of the invention and the methods of the invention. The liposomes typically comprise vesicles containing one or more lipid bilayers enclosing an aqueous interior. Liposome compositions usually include liposomes in a medium, such as an aqueous fluid exterior to the liposome. Liposome lipids can include amphiphilic lipid components that, upon contact with aqueous medium, spontaneously form bilayer membranes, such as phospholipids, for example, phosphatidylcholines. Liposomes also can include membrane-rigidifying components, such as sterols, for example, cholesterol. In some embodiments, liposomes also include lipids conjugated to hydrophilic polymers, such as, polyethylene glycol (PEG) lipid derivatives that may reduce the tendency of liposomes to aggregate and also have other beneficial effects.

In some embodiments, a liposomal composition comprises an ATR inhibitor or a PARP inhibitor encapsulated in a liposome. In some embodiments, a liposomal composition comprises an ATR inhibitor as the only pharmaceutically active agent. In some embodiments, a liposomal composition comprises a PARP inhibitor as the only pharmaceutically active agent. In some embodiments, a liposomal composition comprises an ATR inhibitor and a PARP inhibitor in the same composition.

In some embodiments, a liposomal composition comprises a compound of formula (I) or a pharmaceutically acceptable salt thereof or a PARP inhibitor encapsulated in a liposome. In some embodiments, a liposomal composition comprises a compound of formula (I) or a pharmaceutically acceptable salt thereof as the only pharmaceutically active agent. In some embodiments, a liposomal composition comprises a PARP inhibitor as the only pharmaceutically active agent. In some embodiments, a liposomal composition comprises a compound of formula (I) or a pharmaceutically acceptable salt thereof and a PARP inhibitor in the same composition.

In some embodiments, the liposomal composition comprises a liposome-forming lipid, a cholesterol or a polymer-conjugated lipid. In some embodiments, the liposome-forming lipid comprises one or more phospholipids.

In some embodiments, exemplary liposomal membranes (the bilayer of phospholipids separating the internal aqueous medium from the external aqueous medium) comprises or may be formed from phospholipids, diglycerides, dialiphatic glycolipids, sphingomyelin, egg sphingomyelin (ESM), dihydrosphingomyelin (DHSM), glycosphingolipid, or cholesterol or derivates thereof, or combinations thereof. In some embodiments, the liposome-forming lipid or the liposome lipid comprise phospholipids, diglycerides, dialiphatic glycolipids, sphingomyelin, egg sphingomyelin (ESM), dihydrosphingomyelin (DHSM), glycosphingolipid, or cholesterol or derivates thereof, or combinations thereof.

In general, a variety of liposome lipid components can be used to make the liposomal composition as disclosed herein. Liposome lipid components can include, but are not limited to (a) uncharged lipid components, e.g., cholesterol, ceramide, diacylglycerol, acyl(poly ethers) or alkylpoly(ethers); (b) neutral phospholipids, e.g., diacylphosphatidylcholines, sphingomyelins, and diacylphosphatidylethanolamines, (c) anionic lipids, e.g., diacylphosphatidylserine, diacylphosphatidylglycerol, diacylphosphatidate, cardiolipin, diacylphosphatidylinositol, diacylglycerolhemisuccinate, diaclyglycerolhemigluratate, cholesterylhemisuccinate, cholesterylhemiglutarate, and the like; (d) polymer-conjugated lipids, e.g., N-[methoxy-(poly(ethylene glycol)diacylphosphatidylethanolamine, poly(ethylene glycol)-diacylglycerol, poly(ethylene glycol)-ceramide; and (e) cationic lipids, e.g., 1,2,-diacyl-3-trimethylammonium-propane (DOTAP), dimethyldioctadecylammonium bromide (DDAB), and 1,2-diacyl-sn-glycero-3-ethylphosphocholine. Monoacyl-substituted derivatives of these lipids, as well as di- and monoalkyl-analogs can also be used.

Various liposome lipid components can be selected to fulfill, modify or impart one or more desired functions. Phospholipid can be used as principal vesicle-forming lipid. Inclusion of cholesterol can be useful for maintaining membrane rigidity and decreasing drug leakage. Polymer-conjugated lipids (such as PEG-lipid conjugates) can be used in the liposomal formulation to increase the lifetime of circulation via reducing liposome clearance by liver and spleen, or to improve the stability of liposomes against aggregation during storage, in the absence of circulation extending effect.

In some embodiments, the liposome lipid comprises a phospholipid, a cholesterol, and a PEG-lipid conjugate.

In some embodiments, the phospholipid is phosphatidic acid, phosphatidyl glycerol, phosphatidylcholine, phosphatidylethanolamine, phosphatidylinositol, phosphatidylserine, or mixtures thereof. In some embodiments, the phospholipid is 1,2-dipalmitoyl-sn-glycero-3-phosphocholine (DPPC), dimyristoyl-phosphatidylcholine (DMPC), hydrogenated soy phosphatidylcholine (HSPC), soy phosphatidylcholine (SPC), dimyristoylphosphatidylglycerol (DMPG), disrearoylphosphatidylglycerol (DSPG), 1-palmitoyl-2-oleoyl-sn-glycero-3-phosphocholine (POPC), 1,2-dioleoyl-sn-glycero-3-phosphocholine (DOPC), distearoyl phosphatidylcholine (DSPC), egg yolk phosphatidylcholine (EYPC) or hydrogenated egg yolk phosphatidylcholine (HEPC), sterol modified lipids (SML), cationic lipids and inverse-zwitterlipids. In some embodiments, the phospholipid is distearoylphosphatidylethanolamine (DSPE). In some embodiments, the phospholipid is 1,2-distearoyl-sn-glycero-3-phosphocholine (DSPC).

In some embodiments, phospholipid is a diacylphosphatidylcholine (PC). In some embodiments, PC is derived from natural sources. In some embodiments, PC is derived from synthetic sources.

In some embodiments, the PC is dipalmitoylphosphocholine (DPPC), dimyristoylphosphatidylcholine (DMPC), dimyristoylphosphatidylglycerol (DMPG), distearoylphosphatidylglycerol (DSPG), 1-palmitoyl-2-oleoyl-sn-glycero-3-phosphocholine (POPC), dioleoylphosphocholine (DOPC), distearoyl phosphatidylcholine (DSPC), or dilinoleoylphosphatidylcholine (DLPC). In some embodiments, the PC is soy phosphatidylcholine (SPC), hydrogenated soy phosphatidylcholine (HSPC), egg yolk phosphatidylcholine (EYPC) or hydrogenated egg yolk phosphatidylcholine (HEPC).

In some embodiments, the liposome comprises or is prepared with DSPC, cholesterol, or methoxy-poly(ethylene glycol)-1,2-distearoyl-sn-glyceryl (PEG-DSG), or a combination thereof. In some embodiments, the liposome comprises or is prepared with DSPC, cholesterol, and PEG-DSG.

In some embodiments, the liposome comprises or is prepared with DSPC, cholesterol, or 1,2-dimyristoyl-rac-glycero-3-methylpolyoxyethylene (PEG-DMG), or a combination thereof. In some embodiments, the liposome comprises or is prepared with DSPC, cholesterol, and PEG-DMG.

In some embodiments, the PEG-lipid conjugate is polyethylene glycol phosphatidylethanolamine, polyethylene glycol-diacylglycerol, or polyethylene glycol-ceramide derivative. In some embodiments, the PEG-lipid conjugate is PEG-DSG. In some embodiment, the PEG-lipid is PEG-DSG having a molecular weight from about 550 Daltons to about 5,000 Daltons. In some embodiments, PEG-DSG has a molecular weight of about 550 Daltons, about 600 Daltons, about 700 Daltons, about 800 Daltons, about 900 Daltons, about 1,000 Daltons, about 1,100 Daltons, about 1,200 Daltons, about 1,300 Daltons, about 1,400 Daltons, about 1,500 Daltons, about 1,600 Daltons, about 1,700 Daltons, about 1,800 Daltons, about 1,900 Daltons, about 2,000 Daltons, about 2,100 Daltons, about 2,200 Daltons, about 2,300 Daltons, about 2,400 Daltons, about 2,500 Daltons, about 2,600 Daltons, about 2,700 Daltons, about 2,800 Daltons, about 2,900 Daltons, about 3,000 Daltons, about 3,100 Daltons, about 3,200 Daltons, about 3,300 Daltons, about 3,400 Daltons, about 3,500 Daltons, about 3,600 Daltons, about 3,700 Daltons, about 3,800 Daltons, about 3,900 Daltons, about 4,000 Daltons, about 4,100 Daltons, about 4,200 Daltons, about 4,300 Daltons, about 4,400 Daltons, about 4,500 Daltons, about 4,600 Daltons, about 4,700 Daltons, about 4,800 Daltons, about 4,900 Daltons, or about 5,000 Daltons. In some embodiments, the PEG-DSG is PEG1000-DSG, PEG2000-DSG, or PEG3000-DSG. In some embodiments, the PEG-DSG is PEG2000-DSG.

In some embodiments, the PEG-lipid conjugate is PEG-DMG. In some embodiment, the PEG-lipid is PEG-DMG having a molecular weight from about 550 Daltons to about 5,000 Daltons. In some embodiments, PEG-DMG has a molecular weight of about 550 Daltons, about 600 Daltons, about 700 Daltons, about 800 Daltons, about 900 Daltons, about 1,000 Daltons, about 1,100 Daltons, about 1,200 Daltons, about 1,300 Daltons, about 1,400 Daltons, about 1,500 Daltons, about 1,600 Daltons, about 1,700 Daltons, about 1,800 Daltons, about 1,900 Daltons, about 2,000 Daltons, about 2,100 Daltons, about 2,200 Daltons, about 2,300 Daltons, about 2,400 Daltons, about 2,500 Daltons, about 2,600 Daltons, about 2,700 Daltons, about 2,800 Daltons, about 2,900 Daltons, about 3,000 Daltons, about 3,100 Daltons, about 3,200 Daltons, about 3,300 Daltons, about 3,400 Daltons, about 3,500 Daltons, about 3,600 Daltons, about 3,700 Daltons, about 3,800 Daltons, about 3,900 Daltons, about 4,000 Daltons, about 4,100 Daltons, about 4,200 Daltons, about 4,300 Daltons, about 4,400 Daltons, about 4,500 Daltons, about 4,600 Daltons, about 4,700 Daltons, about 4,800 Daltons, about 4,900 Daltons, or about 5,000 Daltons. In some embodiments, the PEG-DMG is PEG1000-DMG, PEG2000-DMG, or PEG3000-DMG. In some embodiments, the PEG-DSG is PEG2000-DMG.

In some embodiments, PEG in the PEG-lipid conjugate has a molecular weight from about 250 to about 20,000. In some embodiments, PEG in the PEG-lipid conjugate has a molecular weight from about 500 to about 5,000. In some embodiments, PEG in the PEG-lipid conjugate has a molecular weight of about 1000, about 2000, or about 3000.

In some embodiments, the liposome lipid comprises lecithin. In some embodiments, lecithin is a natural lecithin, a hydrogenated natural lecithin, a synthetic lecithin, 1,2-distearoyl-lecithin, dipalmitoyl lecithin, dimyristoyl lecithin, dioleolyl lecithin, 1-stearoyl-2-oleoyl lecithin, or 1-palmitoyl-2-oleoyl lecithin.

In some embodiments, the liposome lipid comprises phytosterol. In some embodiments, the liposome lipid comprises beta-sitosterol.

In some embodiments, the liposome lipid comprises dioleoylphosphatidylethanolamine (DOPE).

In some embodiments, the liposome comprises about 50 mol % to about 70 mol % of DSPC. In some embodiments, the liposome comprises about 55 mol %, about 56 mol %, about 57 mol %, about 58 mol %, about 59 mol %, about 60 mol %, about 61 mol %, about 62 mol %, about 63 mol %, about 64 mol %, or about 65 mol % of DSPC.

In some embodiments, the liposome comprises about 30 mol % to about 50 mol % of cholesterol. In some embodiments, the liposome comprises about 35 mol %, about 36 mol %, about 37 mol %, about 38 mol %, about 39 mol %, about 40 mol %, about 41 mol %, about 42 mol %, about 43 mol %, about 44 mol %, or about 45 mol % of cholesterol.

In some embodiments, the liposome comprises about 0.1 mol % to about 5 mol % of PEG-DSG. In some embodiments, the liposome comprises about 0.1 mol %, about 0.15 mol %, about 0.2 mol %, about 0.25 mol %, about 0.3 mol %, about 0.35 mol %, about 0.4 mol %, about 0.45 mol %, about 0.5 mol %, about 0.6 mol %, about 0.7 mol %, about 0.8 mol %, about 0.9 mol %, about 1.0 mol %, about 1.5 mol %, about 2.0 mol %, about 2.5 mol %, about 3.0 mol %, about 3.5 mol %, about 4.0 mol %, about 4.5 mol %, or about 5.0 mol % of PEG-DSG.

In some embodiments, the molar ratio of DSPC:cholesterol of the liposome is in the range of about 1:1 to about 2:1. In some embodiments, the molar ratio of DSPC:cholesterol of the liposome is in the range of about 14:10 to about 15:10. In some embodiments, the molar ratio of DSPC:cholesterol of the liposome is about 14:10, about 13:10, or about 12:10.

In some embodiments, the molar ratio of cholesterol:PEG-DSG of the liposome is in the range of about 10:1 to about 100:1. In some embodiments, the molar ratio of cholesterol:PEG-DSG of the liposome is about 20:1. In some embodiments, the molar ratio of cholesterol:PEG-DSG of the liposome is about 400:2, about 400:2.5, or about 400:3.

In some embodiments, the molar ratio of DSPC:cholesterol:PEG-DSG of the liposome is about 27:20:1, about 26:20:1, or about 25:20:1.

In some embodiments, the molar ratio of DSPC:cholesterol:PEG-DSG of the liposome is about 58:40:2 or about 59.75:40:0.25.

In some embodiments of the liposomal composition, the therapeutically active agent:liposome lipids molar ratio is in the range of about 0.05 to about 2.0. In some embodiments, the therapeutically active agent:liposome lipids molar ratio is in the range of about 0.1 to about 1.0. In some embodiments, the therapeutically active agent:liposome lipids molar ratio is about 0.2, about 0.3, about 0.4, about 0.5, about 0.6, about 0.7, about 0.8, about 0.9 or about 1.0. In some embodiments, the therapeutically active agent:liposome lipids molar ratio is about 0.3.

In some embodiments of the liposomal composition, the liposome encapsulation efficiency is greater than 80%. In some embodiments, the liposome encapsulation efficiency is greater than 90%. In some embodiments, the liposome encapsulation efficiency is greater than 95%. In some embodiments, the liposome encapsulation efficiency is about 90%, about 91%, about 92%, about 93%, about 94%, about 95%, about 96%, about 97%, about 98%, about 99%, or about 100%.

In some embodiments of the liposomal composition, an average size (mean diameter) of the liposomes is in the range of about 80 nm to about 150 nm. In some embodiments of the liposomal composition, an average size (mean diameter) of the liposomes is in the range of about 80 nm to about 120 nm. In some embodiments, the average size (mean diameter) of the liposomes is about 90 nm, about 95 nm, about 100 nm, about 105 nm, about 110 nm, about 115 nm, or about 120 nm.

In some embodiments, the liposomal composition comprises am ATR inhibitor, liposome lipids, and a pharmaceutically acceptable carrier or excipient. In some embodiments, the liposomal composition comprises a compound of formula (I) or a pharmaceutically acceptable salt thereof, liposome lipids, and a pharmaceutically acceptable carrier or excipient.

In some embodiments, the liposomal composition comprises a PARPi, liposome lipids, and a pharmaceutically acceptable carrier or excipient.

In some embodiments, a pharmaceutically acceptable carrier is a buffer, normal saline, isotonic dextrose, isotonic sucrose, Ringer's solution, or Hanks' solution. A buffer can comprise histidine, glycine, hydroxyethylpiperazine-ethylsulfonate (HEPES), morpholipo-ethylsulfonate (MES), succinate, tartrate, or citrate as buffer substance.

Methods of preparing and characterizing pharmaceutical liposomal compositions are known in the field (see, e.g., Lasic D. Liposomes: From physics to applications, Elsevier, Amsterdam 1993; G. Gregoriadis (ed.), Liposome Technology, 3rd edition, vol. 1-3, CRC Press, Boca Raton, 2006; Hong et al., U.S. Pat. No. 8,147,867, incorporated by reference herein in their entirety for all purposes). For liposome loading techniques, see also, U.S. Pat. Nos. 7,744,921, 7,238,367, 8,329,213, 8,703,181, 10,722,467 and 10,004,759, each of which are herein incorporated by reference in its entirety for all purposes. For various methods of preparing liposomes and encapsulation of therapeutic agents: see, for example, U.S. Pat. Nos. 3,932,657, 4,235,871. 4,311,712, 4,743,449, 4,452,747, 4,830,858, 4,921,757, and 5,013,556, each of which are herein incorporated by reference in its entirety for all purposes. See also Wang, L. et al. Sci Rep. 2023 Feb. 24; 13(1):3226, which is herein incorporated by reference in its entirety for all purposes.

In some embodiments, the liposomal composition is prepared by an active loading technique. In some embodiments, the liposomal composition is prepared by a passive loading technique.

In some embodiments, preparation of the liposomal composition comprises a step of preparing a blank liposome. In some embodiments, the blank liposome is prepared using a thin film technique.

In some embodiments, preparation of the liposomal composition comprises a step of hydrating the blank liposome with a buffer. In some embodiments, the hydrating step is performed with a TEA₈SOS buffer. In some embodiments, the hydrating step is performed with a 1.1N TEA₈SOS buffer.

In some embodiments, preparation of the liposomal composition comprises a step of high-pressure extrusion following the hydrating step.

In some embodiments, preparation of the liposomal composition comprises a step of exchanging an external liposomal buffer. In some embodiments, the external liposomal buffer is exchanged for dextrose buffer. In some embodiments, the external liposomal buffer is exchanged for 18% dextrose buffer.

In some embodiments, preparation of the liposomal composition comprises a step of further exchanging the external liposomal buffer. In some embodiments, the external liposomal buffer is exchanged for HEPES buffered saline (pH 7.4).

In some embodiments, preparation of the liposomal composition comprises a step of actively loading the ATR inhibitor or the PARP inhibitor into the blank liposome. In some embodiments, preparation of the liposomal composition comprises a step of actively loading the compound of formula (I) or a pharmaceutically acceptable salt thereof or the PARP inhibitor into the blank liposome. In some embodiments, the active loading is done on the blank liposome which was hydrated, extruded, and the external liposomal buffer exchanged.

In some embodiments, the ATR inhibitor or the PARP inhibitor is dissolved in a suitable solvent prior to active loading. In some embodiments, the compound of formula (I) or a pharmaceutically acceptable salt thereof or the PARP inhibitor is dissolved in a suitable solvent prior to active loading.

In some embodiments, preparation of the liposomal composition comprises a step of heating the liposome past the phase transition temperature after the ATR inhibitor or the PARP inhibitor is added. In some embodiments, preparation of the liposomal composition comprises a step of heating the liposome past the phase transition temperature after the compound of formula (I) or a pharmaceutically acceptable salt thereof or the PARP inhibitor is added. In some embodiments, the heating is performed while stirring. In some embodiments, heating is performed for about 1 hour.

In some embodiments, the loaded liposomes are cooled and purified. In some embodiments, purification comprises exchanging the external liposomal buffer. In some embodiments, purification comprises purifying the external liposomal buffer to eliminate any un-loaded ATR inhibitor or PARP inhibitor. In some embodiments, purification comprises purifying the external liposomal buffer to eliminate any un-loaded compound of formula (I) or a pharmaceutically acceptable salt thereof or PARP inhibitor.

In some embodiments, the purified loaded liposomes are concentrated to the desired concentration. In some embodiments, the concentrating step is performed using the tangential flow.

In some embodiments, pharmaceutical compositions can comprise nanoparticles. The formation of nanoparticles has been achieved by various methods. Nanoparticles can be made by precipitating a molecule in a water-miscible solvent, and then drying and pulverizing the precipitate to form nanoparticles. (U.S. Pat. No. 4,726,955). Similar techniques for preparing nanoparticles for pharmaceutical preparations include wet grinding or milling. Other methods include mixing low concentrations of polymers dissolved in a water-miscible solution with an aqueous phase to alter the local charge of the solvent and form a precipitate through conventional mixing techniques. (U.S. Pat. No. 5,766,635). Other methods include the mixing of copolymers in organic solution with an aqueous phase containing a colloid protective agent or a surfactant for reducing surface tension. Other methods of incorporating additive therapeutic agents into nanoparticles for drug delivery require that nanoparticles be treated with a liposome or surfactant before drug administration (U.S. Pat. No. 6,117,454). Nanoparticles can also be made by flash nanoprecipitation (U.S. Pat. No. 8,137,699).

The size of the dose for therapeutic purposes of compounds of the invention will naturally vary according to the nature and severity of the conditions, the age and sex of the animal or patient and the route of administration, according to well-known principles of medicine.

Dosage levels, dose frequency, and treatment durations of compounds of the invention are expected to differ depending on the formulation and clinical indication, age, and co-morbid medical conditions of the patient.

### Therapeutic Use

The present disclosure also relates to method for using the combination of the invention for treating various diseases and conditions. In embodiments, the disease or a condition is implicated by one or more abnormal ATR activities. In embodiments, the disease or a condition is implicated by one or more abnormal PARP activities.

The present disclosure provides method for treating or preventing cancer comprising administering to a subject in need thereof an effective amount of the combination of the invention.

In some embodiments, the disease or the condition is cancer. In embodiments, the disease or the condition is selected from Barret's adenocarcinoma; biliary tract carcinomas; breast cancer; cervical cancer; cholangiocarcinoma; central nervous system tumors; primary CNS tumors; glioblastomas, astrocytomas; glioblastoma multiforme; ependymomas; secondary CNS tumors (metastases to the central nervous system of tumors originating outside of the central nervous system); brain cancer; brain tumors; brain metastases; colorectal cancer; large intestinal colon carcinoma; gastric cancer; carcinoma of the head and neck; squamous cell carcinoma of the head and neck; acute lymphoblastic leukemia; acute myelogenous leukemia (AML); myelodysplastic syndromes; chronic myelogenous leukemia; Hodgkin's lymphoma; non-Hodgkin's lymphoma; megakaryoblastic leukemia; multiple myeloma; erythroleukemia; hepatocellular carcinoma; lung cancer; small cell lung cancer; non-small cell lung cancer; ovarian cancer; endometrial cancer; pancreatic cancer; pituitary adenoma; prostate cancer; renal cancer; metastatic melanoma or thyroid cancers.

In some embodiments, the cancer is gastric cancer. In some embodiments, the cancer is gastroesophageal adenocarcinoma.

In some embodiments, the cancer is lung cancer, brain cancer, colorectal cancer, ovarian cancer, or breast cancer. In some embodiments, the cancer is colorectal cancer. In some embodiments, the lung cancer is small cell lung cancer or non-small cell lung cancer. In some embodiments, the breast cancer is triple negative breast cancer. In embodiments, the cancer is ovarian cancer. In some embodiments, the ovarian cancer is high-grade serous ovarian cancer. In some embodiments, the cancer is a solid tumor.

In some embodiments, the effective amount of the combination of the invention is an amount that provides therapeutic synergy between the ATR inhibitor and the PARP inhibitor. In some embodiments, the effective amount of the combination of the invention is an amount that provides therapeutic synergy between the compound of formula (I) or a pharmaceutically acceptable salt thereof and the PARP inhibitor.

In some embodiments, the effective amount of the combination of the invention is an amount that provides therapeutic synergy between the ATR inhibitor, the PARP inhibitor and the additional pharmaceutically active agent, such as an anti-cancer agent.

In some embodiments, the effective amount of the combination of the invention is an amount that provides therapeutic synergy between the compound of formula (I) or a pharmaceutically acceptable salt thereof, the PARP inhibitor and the additional pharmaceutically active agent, such as an anti-cancer agent.

In some embodiments, the methods of the invention provide therapeutic synergy between the ATR inhibitor and the PARP inhibitor. In some embodiments, the methods of the invention provide therapeutic synergy between the compound of formula (I) or a pharmaceutically acceptable salt thereof and the PARP inhibitor.

In some embodiments, the methods of the invention provide greater tumor growth inhibition when compared to monotherapy with the PARP inhibitor. In some embodiments, the methods of the invention provide at least 5% greater tumor growth inhibition when compared to monotherapy with the PARP inhibitor. In some embodiments, the methods of the invention provide at least 10% greater tumor growth inhibition when compared to monotherapy with the PARP inhibitor. In some embodiments, the methods of the invention provide at least 15% greater tumor growth inhibition when compared to monotherapy with the PARP inhibitor. In some embodiments, the methods of the invention provide at least 20% greater tumor growth inhibition when compared to monotherapy with the PARP inhibitor.

In some embodiments, the methods of the invention provide at least 5% greater tumor growth inhibition when compared to niraparib monotherapy. In some embodiments, the methods of the invention provide at least 10% greater tumor growth inhibition when compared to niraparib monotherapy. In some embodiments, the methods of the invention provide at least 15% greater tumor growth inhibition when compared to niraparib monotherapy. In some embodiments, the methods of the invention provide at least 20% greater tumor growth inhibition when compared to niraparib monotherapy.

In some embodiments, the methods of the invention results in improved toxicity profile when one or both of the compound of formula (I) or a pharmaceutically acceptable salt thereof and the PARP inhibitor is in a liposomal composition (same or different), when compared to a method where one or both of the compound of formula (I) or a pharmaceutically acceptable salt thereof and the PARP inhibitor is in liposome-free compositions.

In some embodiments, the methods of the invention results in improved toxicity profile when compared to standard of care of the disease to be treated.

In some embodiments, the methods of the invention do not cause acute neurological toxicity. In some embodiments, the methods of the invention do not cause severe acute neurological toxicity. In some embodiments, the methods of the invention do not cause hematological toxicity. In some embodiments, the methods of the invention do not cause long-term hematological toxicity.

In some embodiments, the methods of the invention do not cause the subject to have pancytopenia. In some embodiments, the methods of the invention protect the subject from pancytopenia. In some embodiments, the methods of the invention do not cause significant reduction in white blood cell count. In some embodiments, the methods of the invention do not cause significant reduction in reticulocytes.

In some embodiments, the methods of the invention do not cause the subject to have mild, moderate or severe anemia. In some embodiments, the methods of the invention do not cause the subject to have mild, moderate or severe reticulocytopenia. In some embodiments, the methods of the invention do not cause the subject to have an absolute reticulocyte count of less than about 10,000/μL, less than about 20,000/μL, less than about 30,000/μL, less than about 40,000/μL, less than about 50,000/μL, or less than about 60,000/μL. The reticulocyte count can be expressed either as a percentage of all red blood cells (RBCs), the absolute reticulocyte count, the corrected reticulocyte count, the reticulocyte production index (RPI) or as the immature reticulocyte fraction (IRF).

In some embodiments, the methods of the invention do not cause the subject to have mild, moderate or severe neutropenia. In some embodiments, the methods of the invention do not cause the subject to have a neutrophil count of less than about 1500/μL, less than about 1000/μL, or less than about 500/μL. In some embodiments, the methods of the invention do not cause the subject to have a neutrophil count of less than a range of about 1000-1500/μL, less than a range of about 500-1000/μL, or less than about 500/μL.

In some embodiments, the methods of the invention do not cause the subject to have mild, moderate or severe leukopenia. In some embodiments, the methods of the invention do not cause the subject to have grade 1, grade 2, grade 3 or grade 4 leukopenia. In some embodiments, the methods of the invention do not cause the subject to have a total leukocyte count of less than about 5,000-17,000/mm³. In some embodiments, the methods of the invention do not cause the subject to have a total leukocyte count of less than a range of about 5,000-11,000/μL. In some embodiments, the methods of the invention do not cause the subject to have a total leukocyte count of less than about 5,000/μL

In some embodiments of the methods of the invention, the ATR inhibitor is administered in a liposomal composition. In some embodiments of the methods of the invention, the compound of formula (I) or a pharmaceutically acceptable salt thereof is administered in a liposomal composition. In some embodiments, the liposomal composition is any one of the liposomal compositions as disclosed herein. In some embodiments, the compound of formula (I) or a pharmaceutically acceptable salt thereof is a compound of Table A, or a pharmaceutically acceptable salt thereof. In some embodiments, the compound of formula (I) or a pharmaceutically acceptable salt thereof is a compound of Table A.

In some embodiments of the methods of the invention, the PARP inhibitor is administered in a liposomal composition. In some embodiments, the liposomal composition is any one of the liposomal compositions as disclosed herein.

In some embodiments of the methods of the invention where the compound of formula (I) or a pharmaceutically acceptable salt thereof and the PARP inhibitor is administered in same or different liposomal composition, the methods provide greater tumor growth inhibition when compared to a method where the compound of formula (I) or a pharmaceutically acceptable salt thereof and the PARP inhibitor is administered in same or different non-liposomal composition.

In some embodiments of the methods of the invention where the compound of formula (I) or a pharmaceutically acceptable salt thereof and the PARPi is administered in the same or different liposomal composition. In some embodiments, the compound of formula (I) or a pharmaceutically acceptable salt thereof and the PARPi administered in the same liposomal composition. In some embodiments, the compound of formula (I) or a pharmaceutically acceptable salt thereof and the PARPi is administered in different liposomal compositions. In some embodiments, the compound of formula (I) or a pharmaceutically acceptable salt thereof is administered in a liposomal composition and the PARPi is administered in liposome-free composition. In some embodiment, administration of the compound of formula (I) or a pharmaceutically acceptable salt thereof and the PARPi is performed simultaneously, sequentially, or continuously, through the same or different routes. In some embodiment, administration of the compound of formula (I) or a pharmaceutically acceptable salt thereof and the PARPi is done on a separate dosing interval.

In some embodiments, the methods of the invention provide therapeutic synergy between the compound of formula (I) or a pharmaceutically acceptable salt thereof, a PARP inhibitor and an additional pharmaceutically active agent for example an anti-cancer agent anti-cancer agent, such as irinotecan. In some embodiments, the methods of the invention provide greater tumor growth inhibition when compared to the respective monotherapies e.g., PARP inhibitor, a compound of formula (I) or the additional pharmaceutically active agent alone. In some embodiments, the methods of the invention provide at least 5% greater tumor growth inhibition with the combination therapy when compared to the respective monotherapies. In some embodiments, the methods of the invention provide at least 10% greater tumor growth inhibition with the combination therapy when compared to the respective monotherapies. In some embodiments, the methods of the invention provide at least 15% greater tumor growth inhibition with the combination therapy when compared to the respective monotherapies. In some embodiments, the methods of the invention provide at least 20% greater tumor growth inhibition with the combination therapy when compared to the respective monotherapies. In some embodiments, the methods of the invention provide at least 30% greater tumor growth inhibition with the combination therapy when compared to the respective monotherapies.

In some embodiments, the methods of the invention provide at least 5% greater tumor growth inhibition when compared to irinotecan monotherapy. In some embodiments, the methods of the invention provide at least 10% greater tumor growth inhibition when compared to irinotecan monotherapy. In some embodiments, the methods of the invention provide at least 15% greater tumor growth inhibition when compared to irinotecan monotherapy. In some embodiments, the methods of the invention provide at least 20% greater tumor growth inhibition when compared to irinotecan monotherapy. In some embodiments, the methods of the invention provide at least 30% greater tumor growth inhibition when compared to irinotecan monotherapy.

In some embodiments, the methods of the invention provide at least 5% greater tumor growth inhibition when compared to niraparib monotherapy. In some embodiments, the methods of the invention provide at least 10% greater tumor growth inhibition when compared to niraparib monotherapy. In some embodiments, the methods of the invention provide at least 15% greater tumor growth inhibition when compared to niraparib monotherapy. In some embodiments, the methods of the invention provide at least 20% greater tumor growth inhibition when compared to niraparib monotherapy. In some embodiments, the methods of the invention provide at least 30% greater tumor growth inhibition when compared to niraparib monotherapy.

In some embodiments, the methods of the invention provide at least 5% greater tumor growth inhibition when compared to a compound of formula (I) monotherapy. In some embodiments, the methods of the invention provide at least 10% greater tumor growth inhibition when compared to a compound of formula (I) monotherapy. In some embodiments, the methods of the invention provide at least 15% greater tumor growth inhibition when compared to a compound of formula (I) monotherapy. In some embodiments, the methods of the invention provide at least 20% greater tumor growth inhibition when compared to a compound of formula (I) monotherapy. In some embodiments, the methods of the invention provide at least 30% greater tumor growth inhibition when compared to a compound of formula (I) monotherapy.

In some embodiments of the methods of the invention, the anti-cancer agent is administered in a pharmaceutical composition. In some embodiments, the pharmaceutical composition is a liposomal composition (e.g., any one of the liposomal compositions as disclosed herein). In embodiments, the anti-cancer agent is administered in a non-liposomal composition.

In some embodiments of the methods of the invention, the compound of formula (I) or a pharmaceutically acceptable salt thereof, the PARP inhibitor, and the additional anti-cancer agent are administered in same or different compositions. In embodiments, the compound of formula (I) or a pharmaceutically acceptable salt thereof, the PARP inhibitor, and the additional anti-cancer agent are administered in same composition. In embodiments, the compound of formula (I) or a pharmaceutically acceptable salt thereof, the PARP inhibitor, and the additional anti-cancer agent are administered in different compositions.

In some embodiments, the compound of formula (I) or a pharmaceutically acceptable salt thereof and the PARPi are administered in liposomal compositions and the additional pharmaceutically active agent such as an anti-cancer agent is administered in a liposome-free composition. In some embodiments, the compound of formula (I) or a pharmaceutically acceptable salt thereof is administered in a liposomal composition and the PARPi and the additional pharmaceutically active agent such as an anti-cancer agent is administered in liposome-free composition. In embodiments, the compound of formula (I) or a pharmaceutically acceptable salt thereof, the PARPi, and the additional pharmaceutically active agent, e.g., anti-cancer agents are administered in liposome compositions.

In some embodiment, administration of the compound of formula (I) or a pharmaceutically acceptable salt thereof, the PARPi, and the additional pharmaceutically active agent e.g., anti-cancer agent is performed simultaneously, sequentially, or continuously, through the same or different routes. In some embodiments, administration of the compound of formula (I) or a pharmaceutically acceptable salt thereof, the PARPi, and the additional pharmaceutically active agent e.g., anti-cancer agent is done on a separate dosing interval.

In some embodiments of the methods of the invention, the compound of formula (I) or a pharmaceutically acceptable salt thereof is administered at a dose in the range of about 1 mg/m² to about 500 mg/m². In one embodiment, the compound of formula (I) or a pharmaceutically acceptable salt thereof is administered at a dose in the range of about 20 mg/m² to about 400 mg/m². the compound of formula (I) or a pharmaceutically acceptable salt thereof is administered at a dose in the range of about 30 mg/m² to about 200 mg/m².

In some embodiments of the methods of the invention, the PARPi is administered at a dose in the range of about 1 mg/m² to about 1000 mg/m². In one embodiment, the PARPi is administered at a dose in the range of about 30 mg/m² to about 500 mg/m². In some embodiments, the PARPi is administered at a dose in the range of about 60 mg/m² to about 300 mg/m².

In some embodiments of the methods of the invention, the anti-cancer agent is administered at a dose in the range of about 1 mg/m² to about 1000 mg/m². In one embodiment, the anticancer agent is administered at a dose in the range of about 50 mg/m² to about 500 mg/m².

In some embodiments of the methods of the invention, niraparib is administered at a dose in the range of about 50 mg to about 300 mg per day. In some embodiments, niraparib is administered at a dose of about 50 mg, about 100 mg, about 150 mg, or about 200 mg per day.

In some embodiments of the methods of the invention, the compound of formula (I) or a pharmaceutically acceptable salt thereof is administered once a day, twice a day, or three times a day. In some embodiments of the methods of the invention, the compound of formula (I) or a pharmaceutically acceptable salt thereof is administered once every 2 days, once every 3 days, once every 4 days, once every 5 days, once every 6 days, or once every 7 days. In some embodiments of the methods of the invention, the compound of formula (I) or a pharmaceutically acceptable salt thereof is administered once a week, once every two weeks, once every three weeks, or once every four weeks.

In some embodiments of the methods of the invention, the PARPi is administered once a day, twice a day, or three times a day. In some embodiments of the methods of the invention, the PARPi is administered once every 2 days, once every 3 days, once every 4 days, once every 5 days, once every 6 days, or once every 7 days. In some embodiments of the methods of the invention, the PARPi is administered once a week, once every two weeks, once every three weeks, or once every four weeks.

In some embodiments of the methods of the invention, the anti-cancer agent is administered once a day, twice a day, or three times a day. In some embodiments of the methods of the invention, the anti-cancer agent is administered once every 2 days, once every 3 days, once every 4 days, once every 5 days, once every 6 days, or once every 7 days. In some embodiments of the methods of the invention, the anti-cancer agent is administered once a week, once every two weeks, once every three weeks, or once every four weeks.

### EXAMPLES

The disclosure now being generally described, it will be more readily understood by reference to the following examples which are included merely for purposes of illustration of certain aspects and embodiments of the present invention, and are not intended to limit the invention.

### Example 1. Synergy Study of PARP Inhibitors and ATR Inhibitors

In vitro combination cell studies were used to measure synergy between different PARP and ATR inhibitors. A panel of nine ovarian cancer cell lines were selected for combination drug screening. Cells were seeded into 96-well plates at their respectively appropriate cell densities (see Table 1) and allowed to adhere for 12-hours. Seeded cells were then treated with niraparib (NIRB) and the ATRi (a compound of formula (I) or berzosertib (VE822)) both as monotherapy and in combination at varying relative molar ratios for 72-hours. Compounds A, B, and C are compounds from Table A. Cell death was assessed using the acid phosphatase assay. Chou and Talalay combination index values were subsequently obtained through inputting dose-effect readouts through CalcuSyn software. See Chou and Talalay Adv. Enzyme Reg. (1984) 22:27-55. See also, U.S. Pat. Nos. 7,850,990 and 9,271,931, each of which is incorporated by reference in its entirety.

TABLE 1
Description of Key Mutations in Cell Lines Tested and Seeding Density

| | | | | | | | | | | | Seeding |
|---|---|---|---|---|---|---|---|---|---|---|---|
| | | | | | | | | | | | Density |
| Cell Line | ATM | ATR | Akt | BRCA1 | BRCA2 | PTEN | CCNE1 | ARID1A | MYC | P53 | (cells/well) |
| ES-2 | | | Akt2 | | Hetloss | | Het-loss | | | Mut | 400 |
| | | | hetloss | | | | | | | | |
| OVCAR8 | | | Akt3 | Hypermeth | | | | Hetloss | Gain | Mut | 1000 |
| | | | gain | | | | | | | | |
| OV90 | | | Akt2 | | | | Gain | | | Mut (ms) | 5000 |
| | | | gain | | | | | | | | |
| COV362 | | | | Mut (ss and | Hetloss | Gain | | Hetloss | Amp | Mut (ms) | 5000 |
| | | | | fs) and | | | | | | and | |
| | | | | hetloss | | | | | | hetloss | |
| KURAMOCHI | | | Akt2/3 | Hetloss | Mut (ns) | Hetloss | Gain | Gain | Amp | Mut (ms) | 3000 |
| | | | gain | | | | | | | | |
| PEO1 | | | | | Y1655*; | | | | | G244D | 3000 |
| | | | | | Y1655F | | | | | | |
| PEO4 | | | | | | | | | | G244D | 3000 |
| UWB 1.289 | | | | | | | | | | | 3000 |
| UWB 1.289 + | | | | | | | | | | | 2500 |
| BRCA1 | | | | | | | | | | | |

The ratios of agents that display synergistic or additive combination effects over concentration ranges were determined using the Chou-Talalay median-effect method. The analysis utilized an equation wherein the dose that causes a particular effect, fₐ, is given by:

D=Dₘ[fₐ/(1−fₐ)]^{1/m}

in which D is the dose of the drug used, fₐ is the fraction of cells affected by that dose, Dₘ is the dose for median effect signifying the potency and m is a coefficient representing the shape of the dose-effect curve (m is 1 for first order reactions).

This equation was further manipulated to calculate a combination index (CI) on the basis of the multiple drug effect equation as described by Chou and Talalay, Adv. Enzyme Reg. (1984) 22:27-55; and by Chou et al., in: Chou and Rideout (eds), Synergism and Antagonism in Chemotherapy. Academic Press: New York 1991:223-244. A computer program for this calculation (CalcuSyn) is found in: Chou et al., Dose-effect analysis with microcomputers: quantitation of ED50, LD50, synergism, antagonism, low-dose risk, receptor ligand binding and enzyme kinetics. CalcuSyn Manual and Software. 1987 Biosoft Cambridge, UK.

The combination index equation was based on the multiple drug-effect equation of Chou-Talalay derived from enzyme kinetic models. An equation determined only the additive effect rather than synergism and antagonism. However, according to the CalcuSyn program, synergism is defined as a more than expected additive effect, and antagonism as a less than expected additive effect. Chou and Talalay in 1983 proposed the designation of CI=1 as the additive effect, thus from the multiple drug effect equation of two drugs:

CI=(D)₁/(Dₓ)₁+(D)₂/(Dₓ)₂ [Eq. 1]

for mutually exclusive drugs that have the same or similar modes of action, and

CI=(D)₁/(Dₓ)₁+(D)₂/(Dₓ)₂+(D₁)(D₂)/(Dₓ)₁(Dₓ)₂ [Eq. 2]

for mutually non-exclusive drugs that have totally independent modes of action. CI<1, =1, and >1 indicates synergism, additive effect, and antagonism, respectively. Equation 1 or Equation 2 dictates that drug 1, (D)₁, and drug 2, (D)₂, (in the numerators) in combination inhibit x % in the actual experiment. Thus, the experimentally observed x % inhibition may not be a round number but most frequently has a decimal fraction. (Dₓ)₁ and (Dₓ)₂ (in the denominators) of equations 1 and 2 are the doses of drug 1 and drug 2 alone, respectively, inhibiting x %.

For simplicity, mutual exclusivity is usually assumed when more than two drugs are involved in combinations (CalcuSyn Manual and Software; Cambridge: Biosoft 1987).

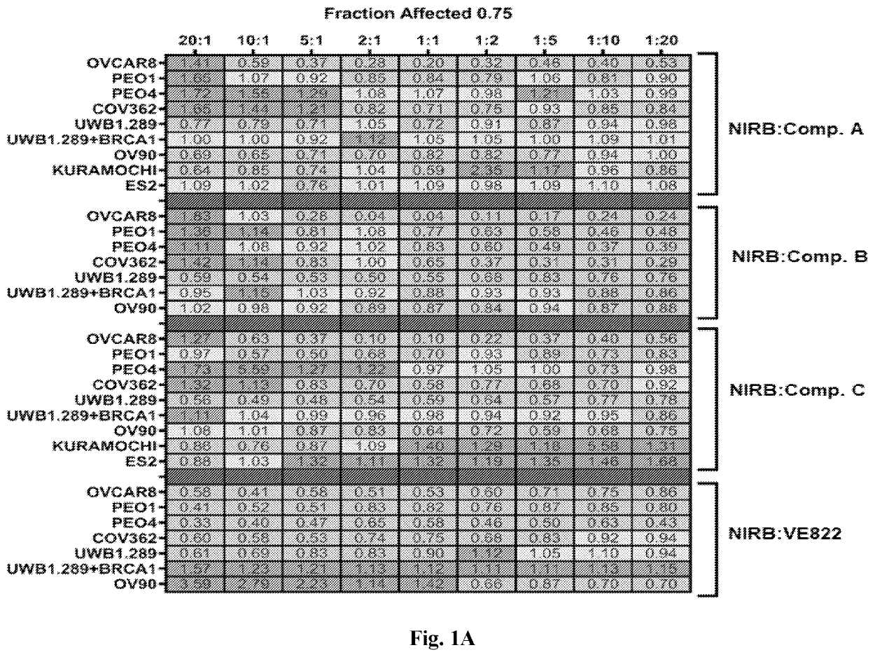

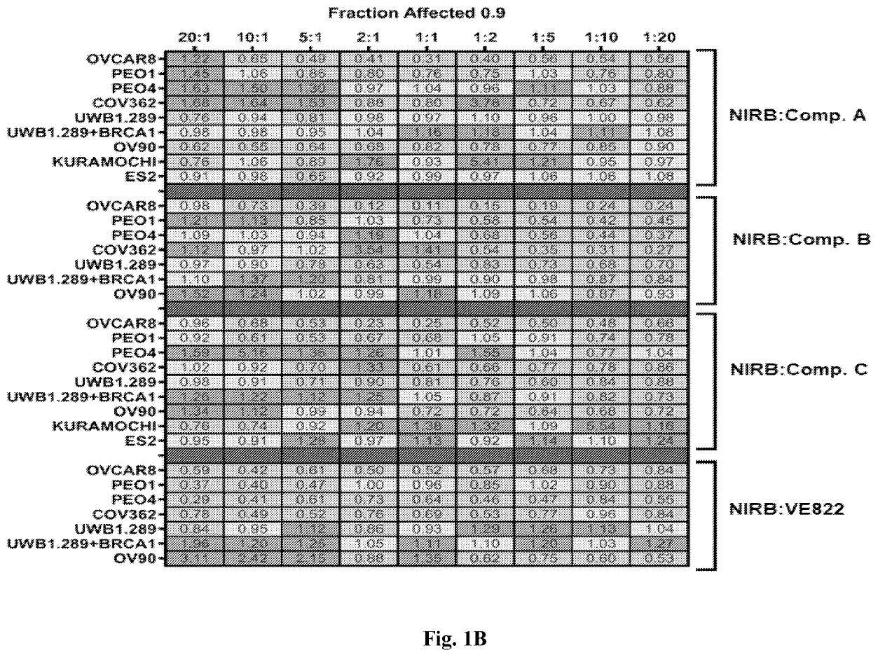

The underlying experimental data are generally determined in vitro using cells in culture or cell-free systems. CI is plotted as a function of the fraction of cells affected (fₐ) as shown in FIGS. 1A-1B which, as explained above, is a surrogate parameter for concentration range.

In some embodiments, useful combinations of agents are those that display synergy or additivity over a substantial range of fₐ values. In some embodiments, useful combinations of agents can be selected that display synergy over at least 5% of the concentration range wherein greater than 1% of the cells are affected, i.e., an fₐ range greater than 0.01. In some embodiments, useful combinations exhibit a favorable CI at a larger portion of overall concentration; for example, 5% of an fₐ range of 0.2-0.8. In some embodiments, 10% of the fₐ range exhibits a favorable CI. In some embodiments, 20% of the fₐ range exhibits a favorable CI. In some embodiments, combinations that provide over 50% or over at least 70% of the fₐ range of 0.2 to 0.8 are useful in the methods and combinations disclosed herein.

Combinations that display synergy over a substantial range of fₐ values may be re-evaluated at a variety of agent ratios to define the optimal ratio to enhance the strength of the non-antagonistic interaction and increase the fₐ range over which synergy is observed.

While it would be desirable to have synergy over the entire range of concentrations over which cells are affected, it has been observed that in many instances, the results are considerably more reliable in an fₐ range of 0.2-0.8.

In some embodiments, the synergy exhibited by combinations of the invention is set forth to exist within the range of 0.01 or greater. In some embodiments, the combination of the invention exhibits synergy when fₐ range is between 0.2-0.8.

The optimal combination ratio may be further used as a single pharmaceutical unit to determine synergistic or additive interactions with a third agent. In addition, a three-agent combination may be used as a unit to determine non-antagonistic interactions with a fourth agent, and so on.

As set forth above, the in vitro studies on cell cultures will be conducted with “relevant” cells. The choice of cells will depend on the intended therapeutic use of the agent. Only one relevant cell line or cell culture type need exhibit the required non-antagonistic effect in order to provide a basis for the compositions to come within the scope of the invention.

Heat map with CI values of various niraparib (“NIRB”) and ATRi (Compounds A, B, and C, and VE822) combinations at 0.75 fraction affected and 0.9 fraction affected are shown in FIG. 1A and FIG. 1B, respectively. CI values less than 0.9 are described as synergistic, between 0.9 and 1.1 as additive, and greater than 1.1 as antagonistic.

### Example 2. In Vitro Combination Mechanistic Studies

Cellular mechanism studies were conducted on the selected panel of ovarian cancer (“OC”) cell lines. OC cells were seeded onto 96-well plates and allowed to adhere for 24-hours. Niraparib (“NIRB”) and ATRi were then added at their respective IC₅₀'s to the adhered cells. All treatments were tested both as monotherapy and at previously determined synergistic and antagonistic combination drug ratios. Plates were then incubated at 37° C. and 5% CO₂ for 72-hours. Treatments were then taken off the plates followed by triple rinse with tris-buffered saline (TBS). Cells were then set with 3% formaldehyde for 20 minutes before permeabilization with 0.2% triton X-100 in TBS for 5 minutes prior to blocking with 5% BSA in TBST for 12-hours at 4° C. Cells were then incubated with either Phospho-Histone H2A.X (Cell Signaling Technology, Danvers, Massachusetts, USA) and Rad51 (GeneTex, San Antonio, Texas, USA) primary antibodies or Phospho-Akt2 (Millipore Sigma, Darmstadt, Germany) and Cyclin E1 (Abcam, Boston, USA) multiplex pairs for 12-hours at 4° C. Resultant cells were then stained with secondary antibodies conjugated to either Alexa Fluor™ 647 or Alexa Fluor™ Plus 555. Cell nucleus and structure were stained for visualization with SYTO™ 9 Green Fluorescent Nucleic Acid Stain and Alexa Fluor™ 350 Phalloidin Dye (Thermo Fisher Scientific, Waltham, MA, USA) respectively. Cells were then imaged and analyzed on the Agilent Cytation 5 (Santa Clara, California).

### Example 3. Preparation of Liposomal Formulation

Formulation A. Blank liposomes containing DSPC:cholesterol:PEG-DSG at relative molar ratios of 58:40:2 was formulated using the thin film technique followed by 1.1N TEA₈SOS buffer hydration and high-pressure liquid extrusion. Prior to immediate drug loading, the external liposomal buffer was exchanged for 18% dextrose buffer.

### Liposome Loading

a) ATR inhibitors: the compounds of formula (I) were dissolved in DMSO, followed by active loading into blank liposomes, in the presence of citrate buffer (pH range of 4-6), to a final concentration of 2% v/v DMSO.
b) Niraparib: the external liposomal buffer of the blank liposomes was further exchanged from 18% dextrose buffer to HEPES (4-(2-hydroxyethyl)-1-piperazineethanesulfonic acid) buffered saline (pH 7.4) before addition of niraparib dissolved in DMSO at a 4% v/v concentration.

Drug added liposomes were heated past the phase transition temperature (i.e., 65° C.) and stirred for 1 hour. Resultant drug loaded liposomes were then cooled on ice for 5 minutes before further purification of external, un-loaded drug (in external liposomal medium) using tangential flow against HEPES buffered saline. Liposomes were then concentrated to the desired drug concentration using tangential flow.

Drug loading was assessed using HPLC-UV.
a) ATR inhibitors of formula (I) were extracted from intact liposomes for HPLC-UV.
b) Niraparib is extracted from intact liposomes by a 1000-fold dilution in methanol. Resulting samples are sonicated for 20-minutes before being centrifuged at 5000 RPM for 20-minutes. 1 mL of supernatant is then collected and detected via HPLC-UV. Lipid analysis is conducted by ELSD.

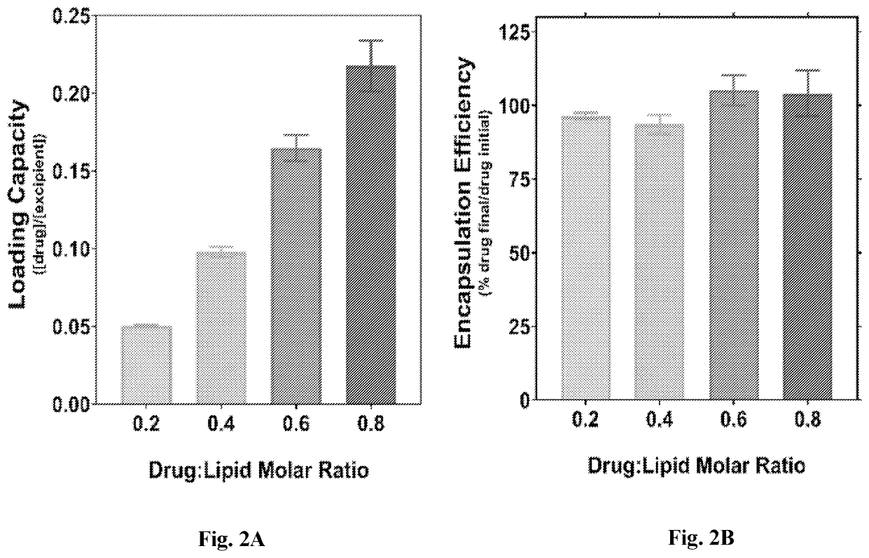

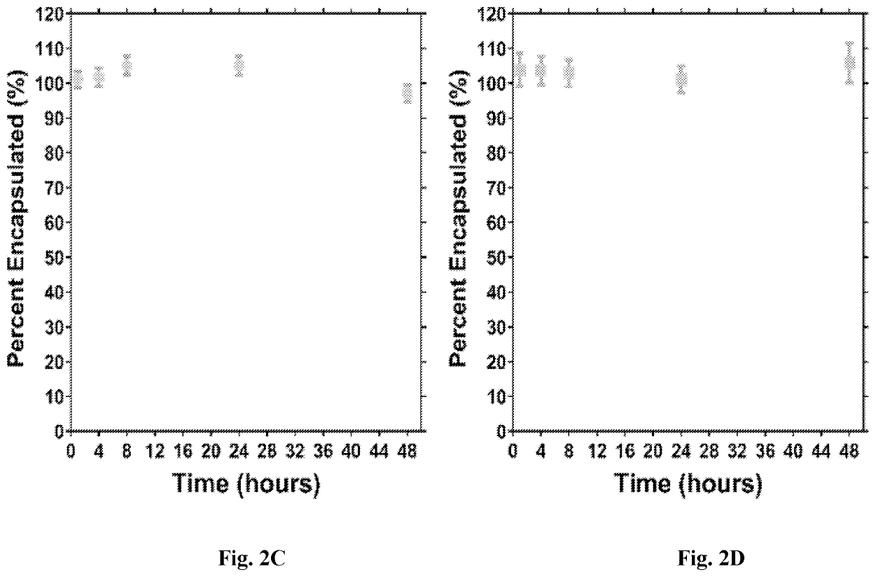

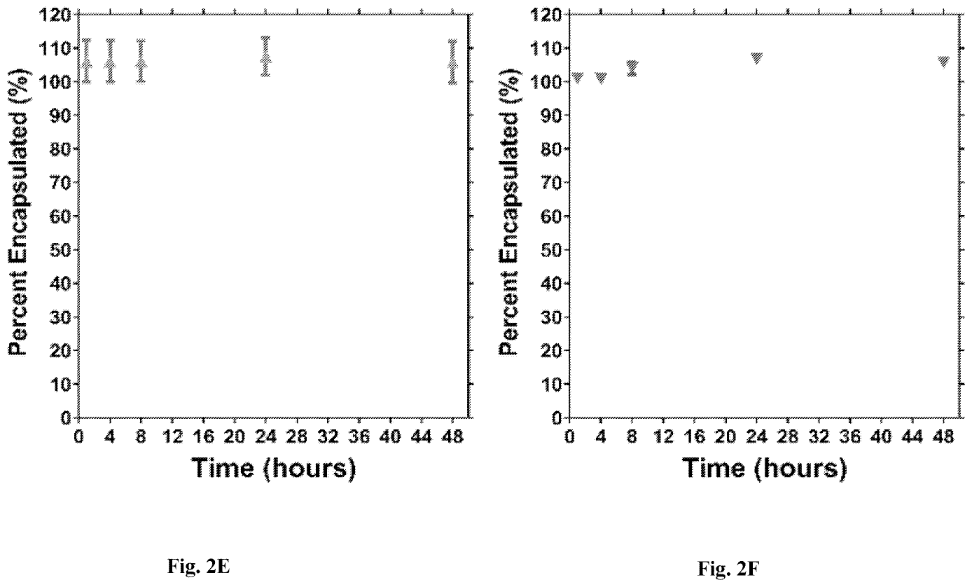

FIG. 2A shows loading capacity of niraparib at various drug:lipid molar ratio. FIG. 2B shows encapsulation efficiency for niraparib at various drug:lipid molar ratio. FIGS. 2C-2F shows in vitro release of niraparib from liposomal formulation at 0.2, 0.4, 0.6, and 0.8 drug:lipid ratio, respectively.

Drug loading and particle stability of niraparib liposomes was assessed. Table 2 summarizes characteristics of niraparib liposomal formulations. TEA₈SOS core liposomes readily facilitated both ATRi and niraparib loading, resulting in pronounced stability.

TABLE 2
Characterization of niraparib liposomal formulation

| Loading | | Loading | | |
|---|---|---|---|---|
| Drug:Lipid | Encapsulation | Capacity | | |
| Molar Ratio | Efficiency (%) | (mg/mg) | Size (nm) | PDI |
| 0.2 | 96 ± 1.2 | 0.03 ± 0.24E−3 | 104.4 ± 32.8 | 0.069 |
| 0.4 | 94 ± 3.2 | 0.06 ± 2.1E−3 | 106.4 ± 35.3 | 0.084 |
| 0.6 | 105 ± 5.2 | 0.08 ± 1.4E−3 | 110.7 ± 29.4 | 0.053 |
| 0.8 | 104 ± 7.8 | 0.10 ± 7.5E−3 | 112.5 ± 29.7 | 0.086 |

PDI = polydispersity index

Formulation B. Formulation B has the same lipids as Formulation A, DSPC:cholesterol:PEG-DSG, but with molar ratios of 59.75:40:0.25.

### Example 4. Drug Tolerance Studies

Maximum tolerated dose studies were conducted on healthy, 6-8 week-old female athymic nude mice (Jackson Laboratories). For baseline monotherapy tolerability, 60 mg/kg of niraparib both in free and liposomal form was IV administered to nude mice at a dosing schedule of every other day followed by a two-day holiday (i.e., three days per week) for 4-weeks (n=5 for each cohort). Free drug was supplied in hydroxybetacyclodextrin in HEPES Buffered Saline with 5% dextrose, pH adjusted to 7.4. Study mice were then sacrificed at 24-hours post final injection with endpoint blood collection by cardiac puncture. Complete blood count (CBC) was then conducted on a Vetscan HM5 hematology analyzer (Zoetis, New Jersey, USA).

This study was conducted over a 4-week period to better capture hematopoietic turnover. Athymic nude mice were selected due to the presence of normal levels of neutrophils, monocytes, and red blood cells on top of a reduced number of non-functional lymphocytes. The immunocompromised nature of athymic nude mice allowed for better comparative translation of dose sensitivity to fully immunocompromised mice (i.e. NSG) that are required for xenograft studies.

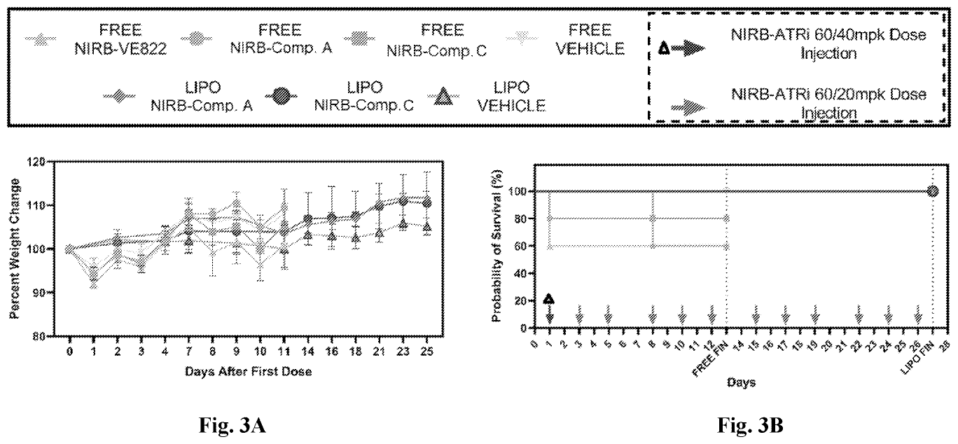

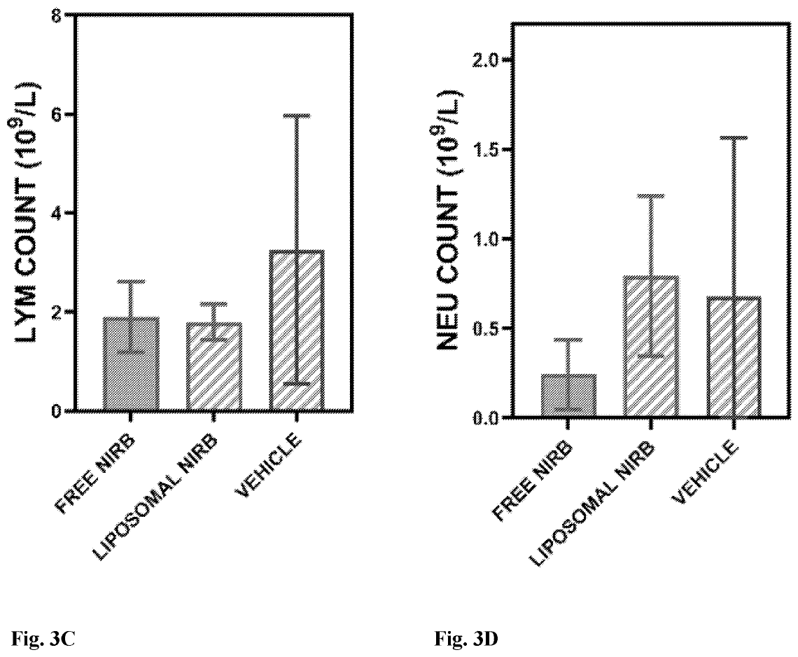

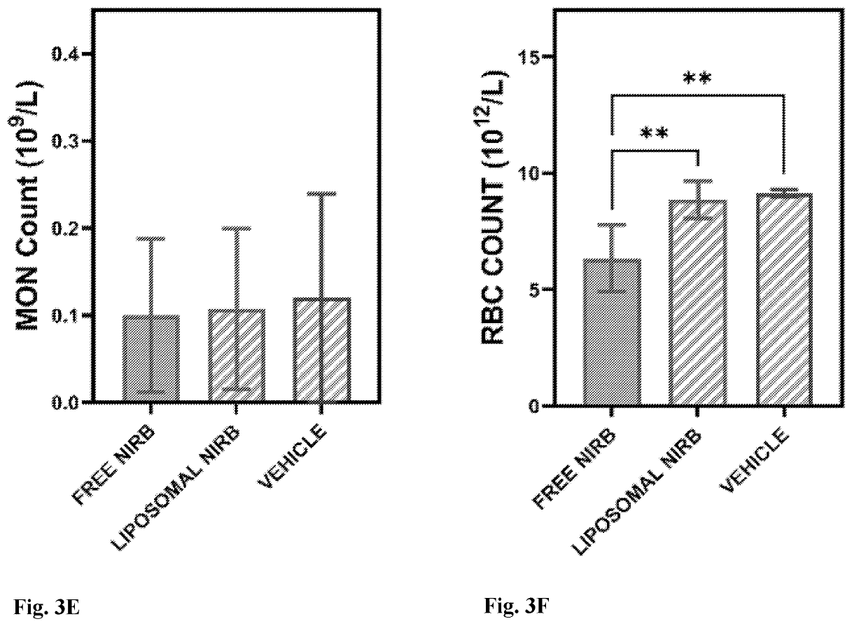

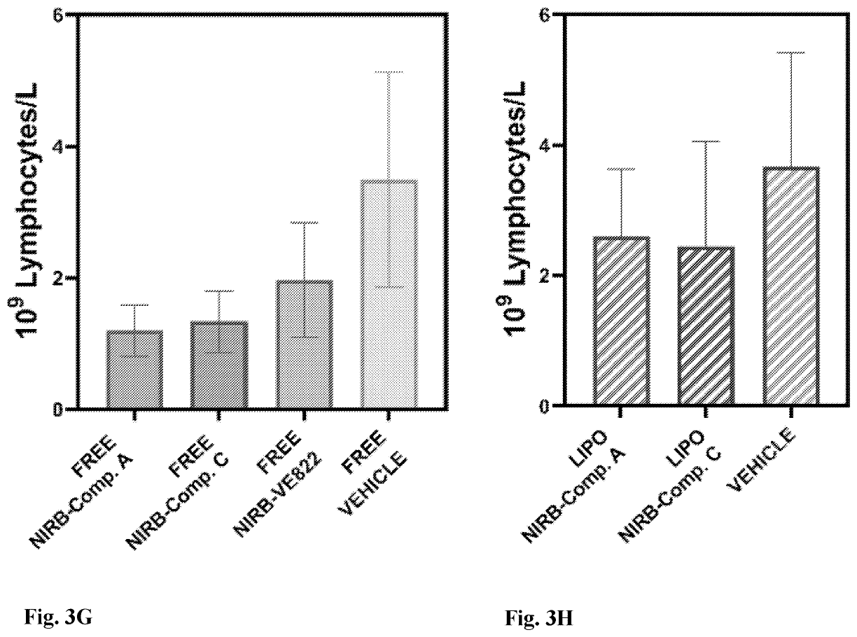

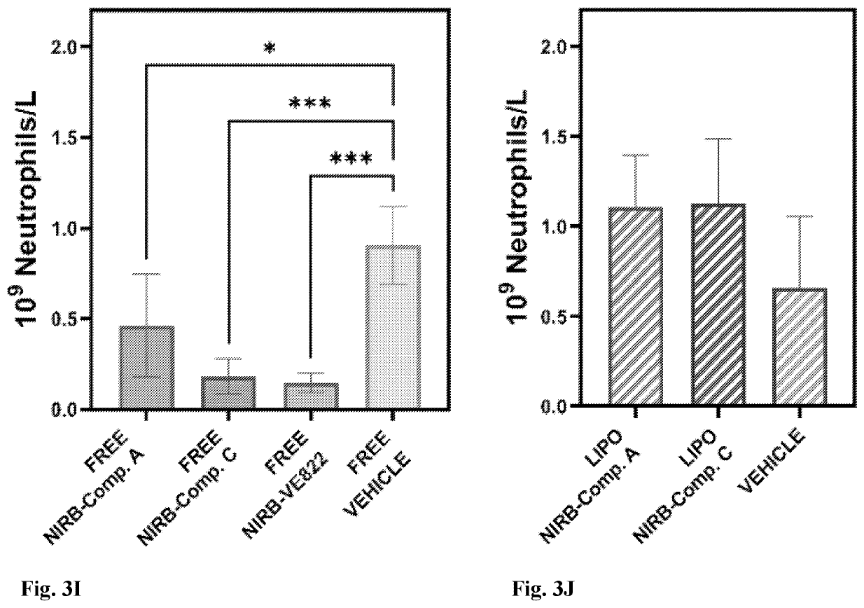

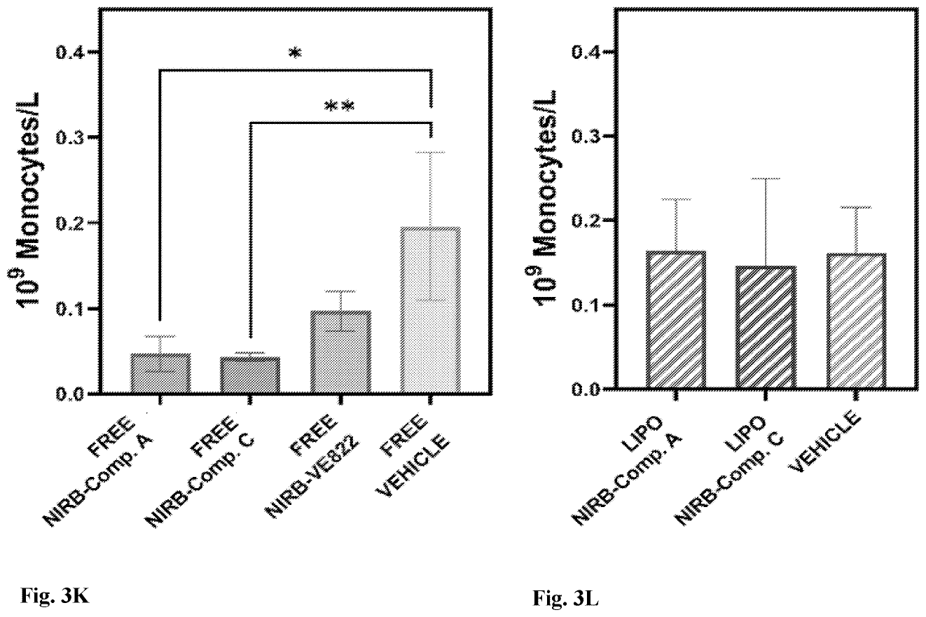

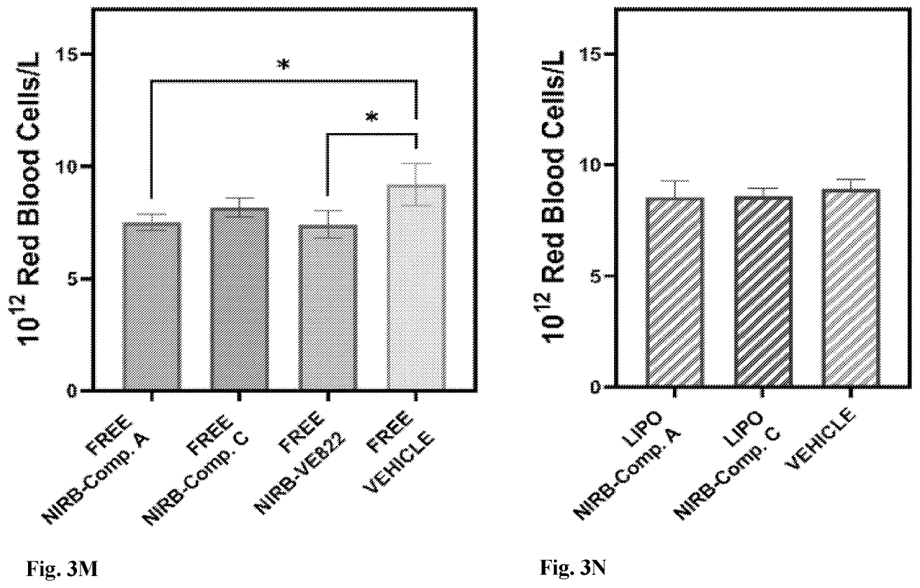

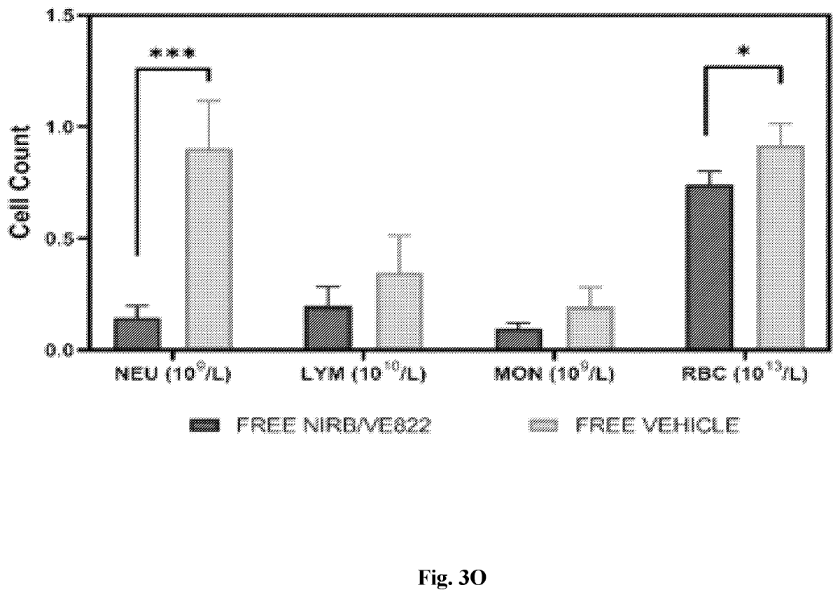

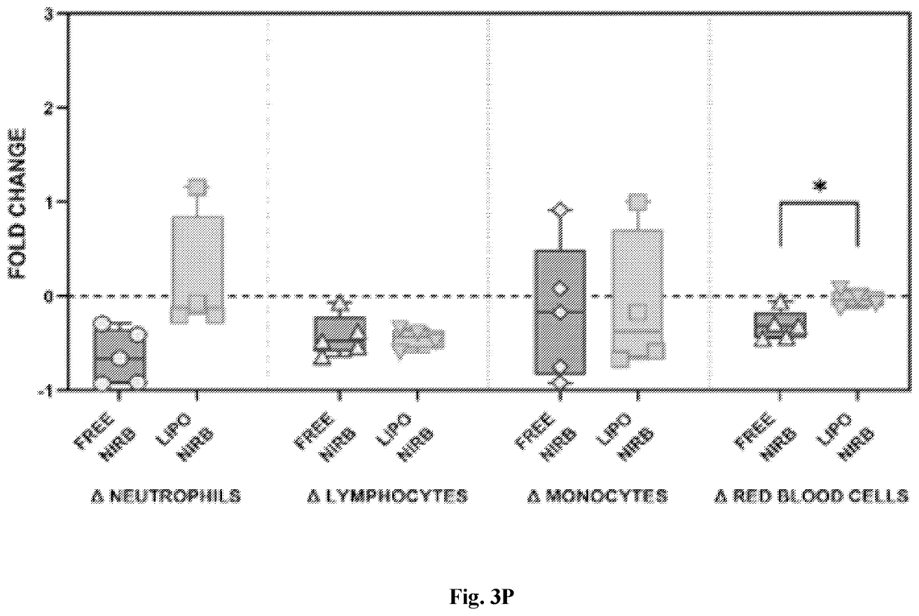

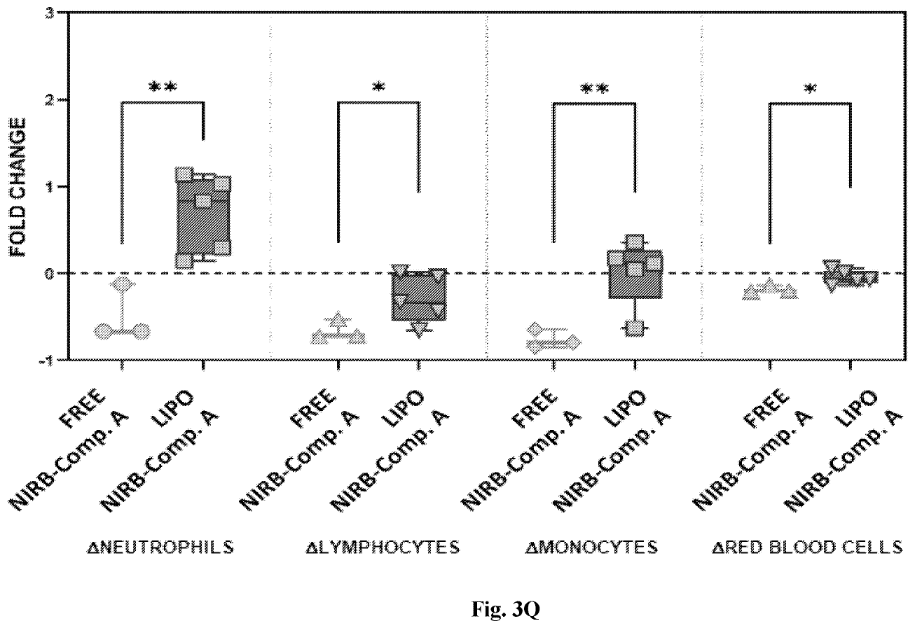

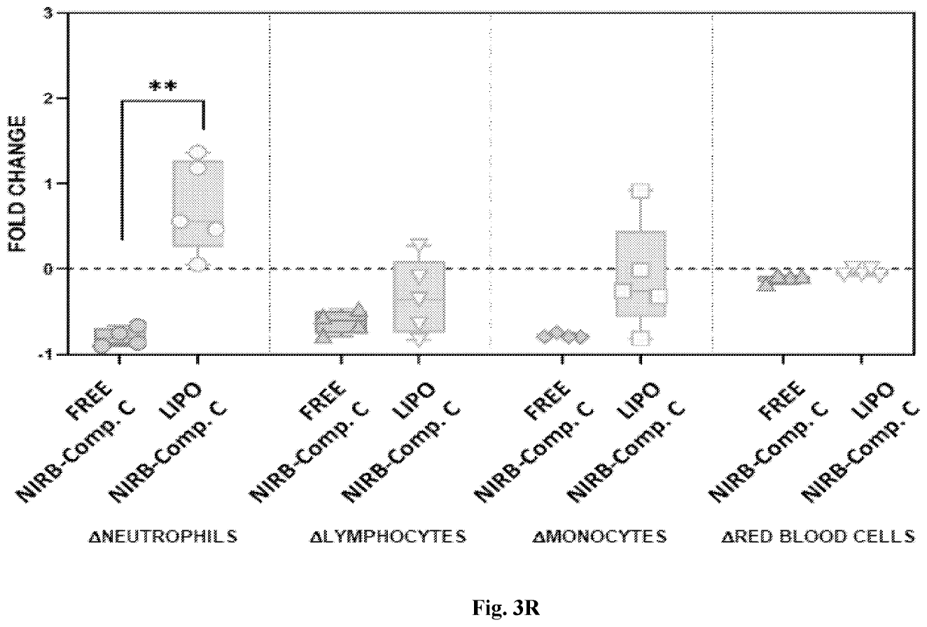

Results for baseline monotherapy: niraparib 60 mg/kg monotherapy in free form (FREE-NIRB) given chronically over 4-weeks at intervals of three-times-weekly (i.e., Monday, Wednesday, and Friday with weekend holiday) was found to cause significant hematological toxicity in the form of macrocytic anemia. Conversely, the liposomal niraparib cohort (LIPO-NIRB) showed no significant changes in blood hematology (i.e., lymphocyte, neutrophils, monocytes, and RBC) when compared to vehicle control. See FIGS. 3C-3F.

Combination therapy tolerability studies were conducted similarly wherein mice were administered either free or liposomal niraparib at 60 mg/kg in combination with ATR inhibitors of formula (I) (Compounds A and C) or berzosertib (VE822) at 20 mg/kg. Mice were administered the combination at the same schedule described above (n=5 for each cohort). Significant acute mortalities resulted in early termination of the study. All cohorts were sacrificed 24-hours after the sixth dose with endpoint blood drawing and CBC analysis as described above.

Results for combination therapy: Addition of ATRi onto the niraparib (NIRB) monotherapy regimens in either free or liposomal forms showed that the NIRB-ATRi combination could only be chronically tolerated in liposomal form. FREE NIRB-ATRi (Compound A, Compound C, and positive control NIRB (VE-822)) administered at a 60/40 mg/kg dose respectively resulted in a 10-20% acute mortality rate secondary to systemic shock within 1-hour of intravenous injection with the benchmark VE-822 cohort showing the worst overall presentation (i.e., acute hyperactivity followed by prolonged lethargy). In contrast, the liposomal combination cohorts (LIPO NIRB-Comp. A and LIPO NIRB-Comp. C) exhibited no signs of acute systemic adverse drug reactions (ADRs). See FIGS. 3G-3N.

A sliding scale dose reduction was applied to all NIRB-ATRi combinations wherein the all-repeat doses were given at 60/20 mg/kg respectively to improve free drug tolerability. Mice treated with free NIRB (VE-822) exhibited significant neutropenia and macrocytic anemia compared to Vehicle control mice. Furthermore, mice treated with FREE NIRB-Comp. A and FREE NIRB-Comp. C had reduced levels of neutrophils, lymphocytes, monocytes, and RBC compared to mice treated with LIPO NIRB-Comp. A and LIPO NIRB-Comp. C, respectively.

FIG. 3A and FIG. 3B show systemic toxicity in drug administered mice expressed as weight change and survival over a 2-4 weeks study duration, respectively. FIGS. 3C-3N show results of CBC analysis after free niraparib monotherapy, liposomal niraparib monotherapy, and free or liposomal combination therapy with niraparib and Compound A or Compound C, over a 2-4 weeks study duration. FIGS. 3O-3R shows fold change in CBC parameters when compared to relative controls.

### Example 5. In Vivo Study of the Combination of an ATR Inhibitor and a PARP Inhibitor

### Pharmacokinetic Studies

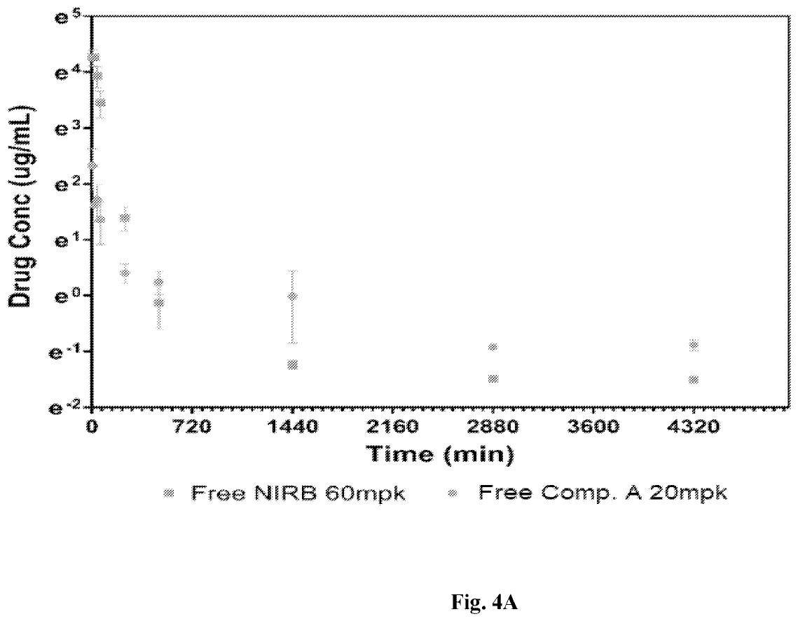

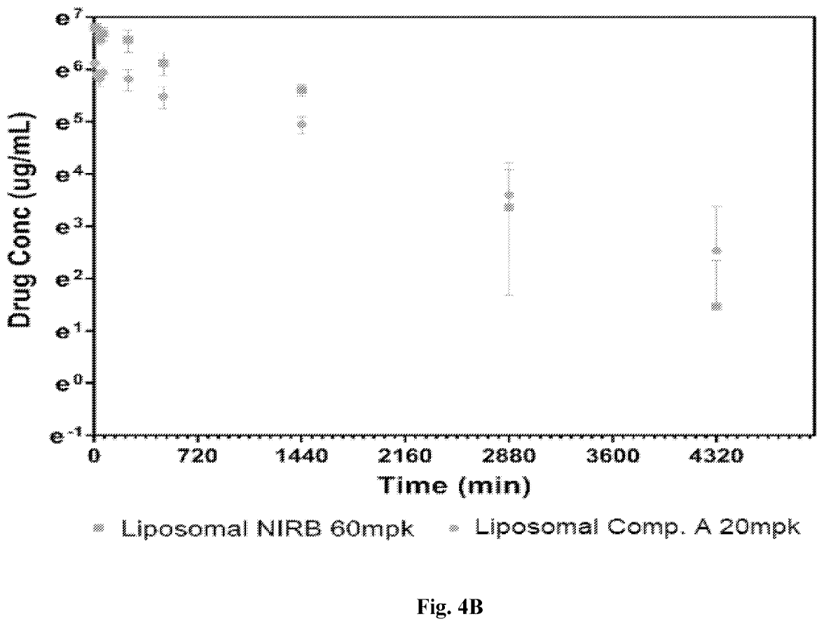

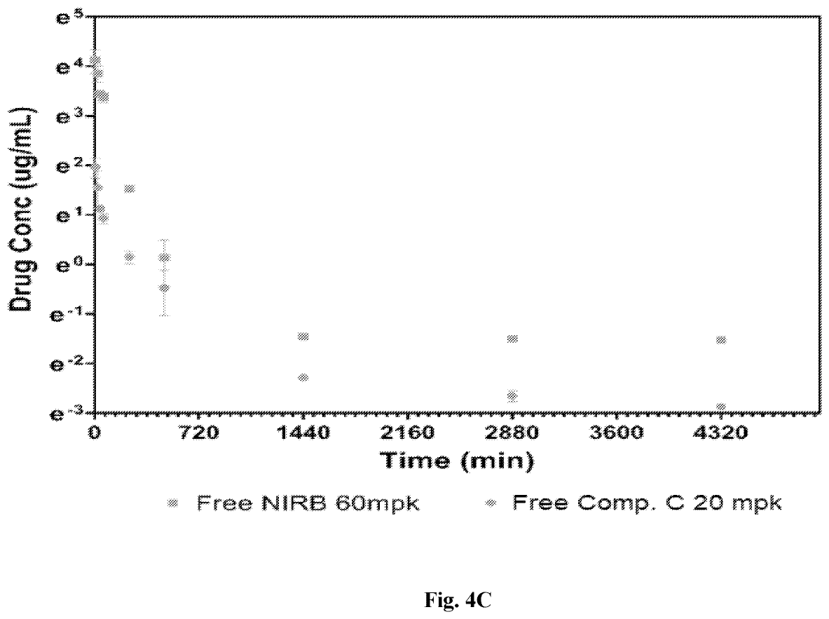

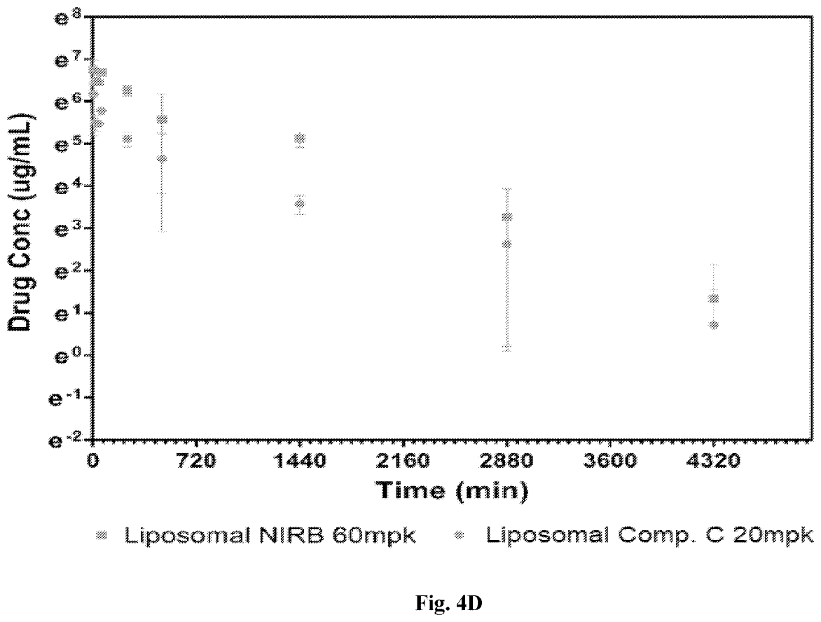

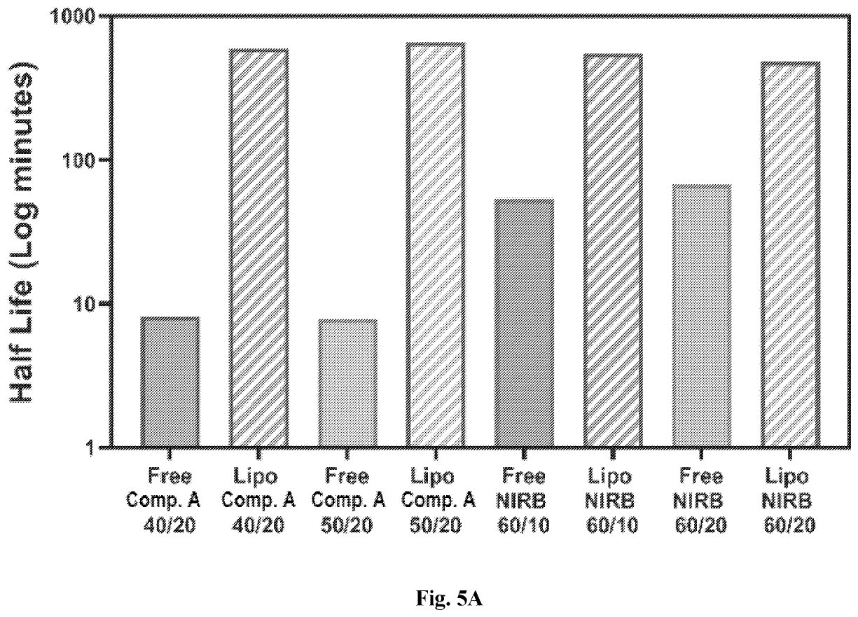

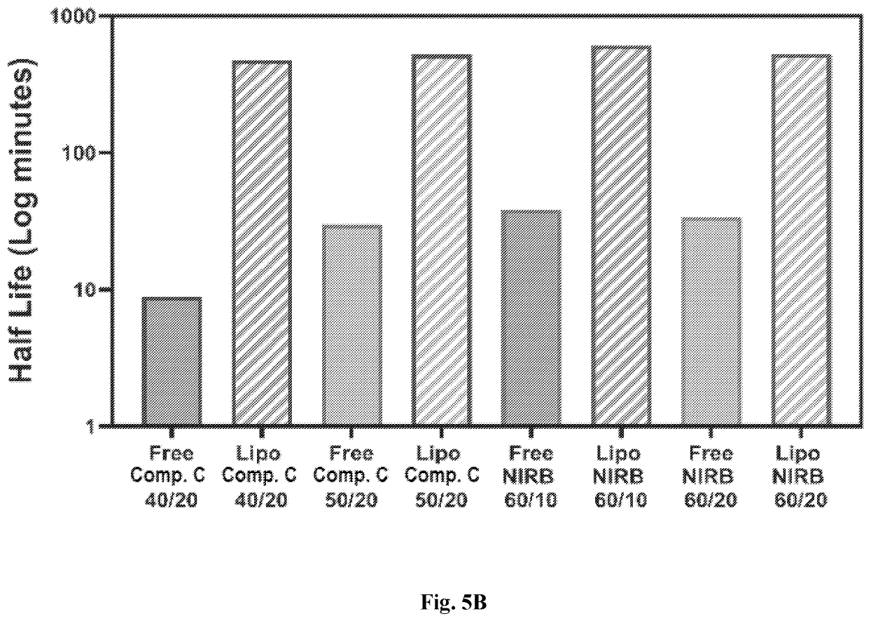

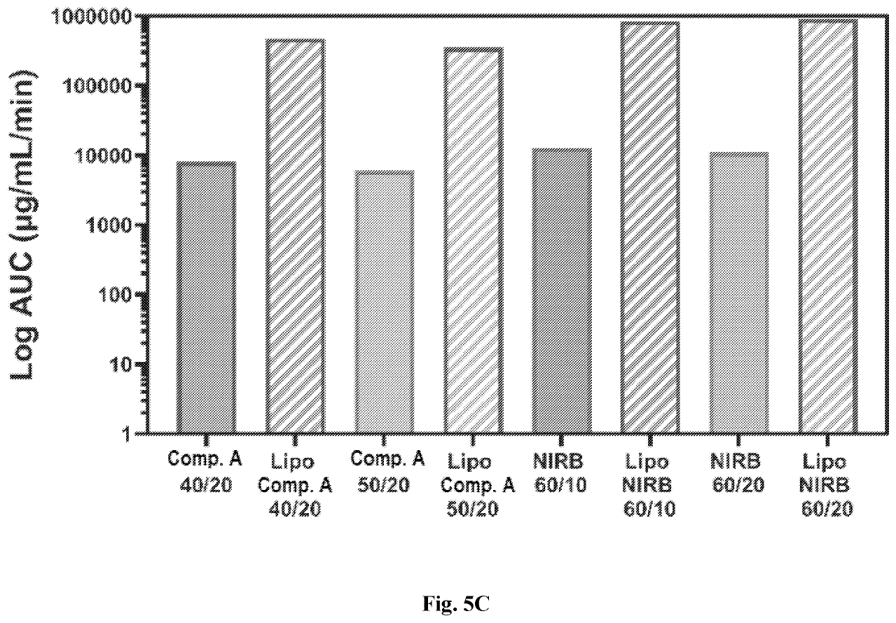

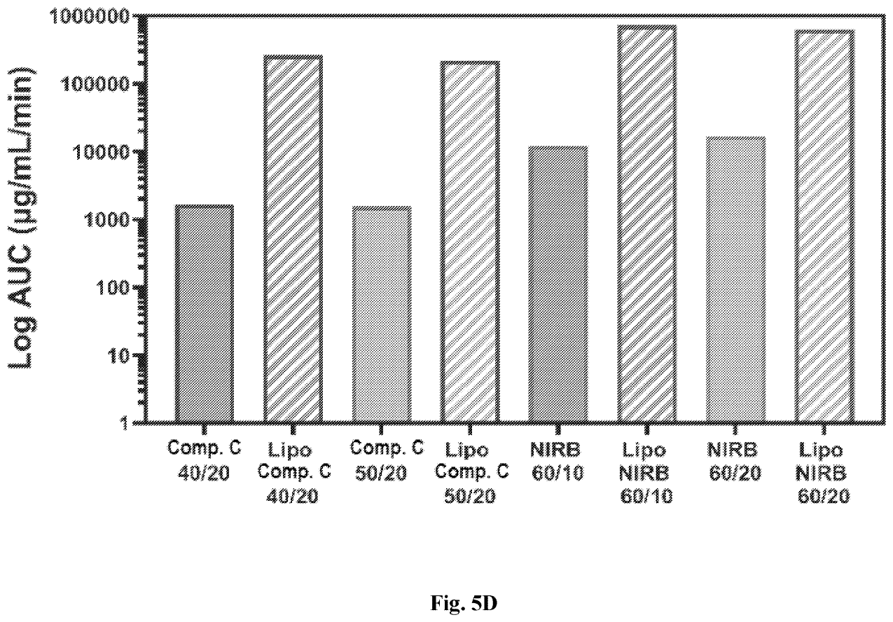

Pharmacokinetic studies were used to measure in vivo activity. The pharmacokinetic profile of liposomal niraparib and ATRi of formula (I) combination in liposomal formulation and non-liposomal formulation was assessed at various dose changes in healthy, 6-8-week-old female athymic nude mice. A combination of ATRi and niraparib in liposomal formulations or a combination of ATRi and niraparib in non-liposomal formulations were administered intravenously as bolus dose to mice at either 40/20 mg/kg, 50/20 mg/kg, 60/20 mg/kg, or 60/10 mg/kg (niraparib/ATRi; n=3 mice per group). Serial blood samples were taken from the flank of mice at timepoints of 0 minute, 5 minutes, 20 minutes, 40 minutes, 1 hour, 4 hours, 8 hours, 24 hours, 48 hours, and 72 hours. FIGS. 4A-4D show blood concentrations of niraparib and ATRi (Compounds A and C) over time. Half lives (FIGS. 5A-5B) and cumulative AUCs (FIGS. 5C-5D) were then calculated using the model of best fit (i.e., a 3-compartment model for free drug and a 1-compartment model for liposomal drug).

Analysis of whole blood drug extract revealed that liposomal formulation of the NIRB/ATRi combinations resulted in a substantial increase in AUC relative to free drug combinations. Furthermore, pharmacokinetic analysis of the free drug combinations revealed that elevations in administered free NIRB markedly slowed down the systemic α-half life of Compound C. This observed drug-drug interaction effect was eliminated in the liposomal cohort wherein the rate of elimination from the central blood compartment of both drugs was significantly slowed to match the predominant elimination characteristics of the liposome.

### Pharmacodynamic Studies

A pharmacodynamic study to elucidate the molecular inhibition kinetics of liposomal niraparib (Formulation A) and liposomal Compound A (Formulation A) when combined with subtherapeutic irinotecan revealed that liposomal encapsulation of niraparib and Compound A allowed for sustained inhibition of the molecular targets at the tumor site. Administration of liposomal niraparib was found to result in early peak of γH2AX indicating DNA damage while liposomal Compound A was found to elicit long acting, later peaking γH2AX indicating DNA damage.

Furthermore, administration of combinations of liposomal niraparib and liposomal Compound A allowed for an even greater sustained DNA damaging effect, mirroring the tumor reduction results shown below in Example 6.

### Example 6. Efficacy of Liposomal Combinations of PARP Inhibitor and ATR Inhibitor

Tumor efficacy studies were used to compare the efficacy of combinations of PARPi (niraparib) and ATRi (Compounds A and C) in liposomal formulations and in non-liposomal (liposome-free) formulations.

### Repeat Administration of Liposomal Formulations Vs Non-Liposomal (Liposome-Free) Formulations

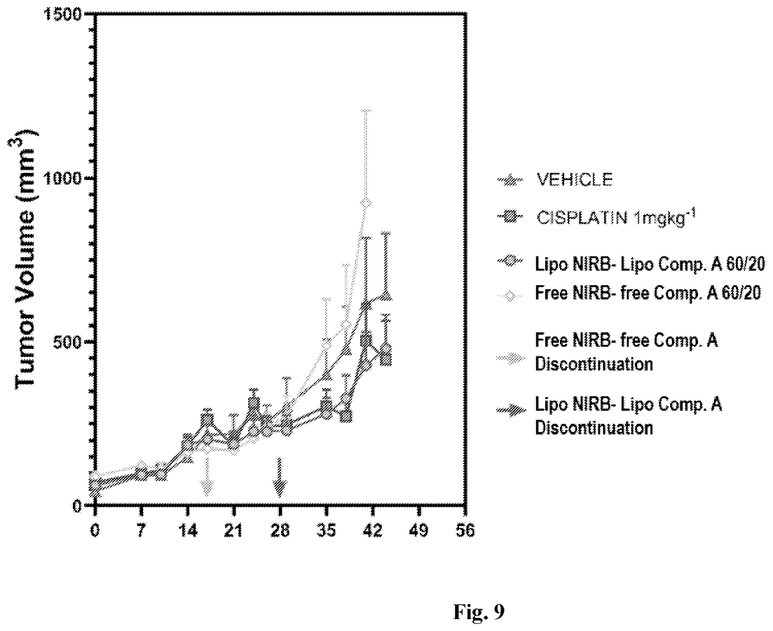

Initial studies to elucidate the tumor reduction potential of the niraparib (NIRB)/Compound A combination as either free or liposomal form revealed that repeat administration of the free drug NIRB/Compound A combination (60 mg/kg and 20 mg/kg, respectively) was unsustainable due to gross mortality upon rechallenge—resulting in early treatment discontinuation and no observation of tumor reduction efficacy (FIG. 9). In contrast, repeat administration with liposomal NIRB/liposomal Compound A (60 mg/kg and 20 mg/kg, respectively) was sustainable over a four-week duration (NIRB and Compound A were in separate liposomal compositions).

### Triple Negative Breast Cancer

Subcutaneous tumor models were initiated on 8-12 week old, female athymic NUDE mice. A cell suspension containing 1 million TNBC MDA-MB-436 cells in 1:1 PBS:Matrigel was subcutaneously injected into the hind flank of the mice. Tumors were measured twice weekly by digital caliper and reported as volume, calculated as V=π×l×w\hat{ }2 wherein l and w denote the longest and shortest tumor diameter respectively. Body weight was measured qd×5, then biweekly to end point. Engrafted mice were randomized into cohorts for treatment at an average tumor size of 100-150 mm³.

Mice were organized into appropriate groups (9 mice pre group) consisting of control (empty vehicle) and treatment groups including free niraparib (40 mg/kg QD), and niraparib (40 mg/kg QD) in combination with liposomal Compound A at two different doses (20 mg/kg or 40 mg/kg, every other day). Dosing Schedule is shown in FIG. 6. Mice were administered niraparib orally, and injected intravenously for liposomal compounds, with the required volume of sample to administer the prescribed dose to the animals based on individual mouse weights on dosing days. Animals were weighed and monitored for survival and in-life observations were collected at the time of weight measurement. Mice were monitored individually to endpoint which was either an individual tumor weight of 2000 mm³ or 60 days, whichever came first.

FIG. 6 shows change in mean tumor volume when free niraparib is administered at 40 mg/kg QD (Grp 2), free niraparib and liposomal Compound A is administered at 20 mg/kg QOD (Grp 5), free niraparib and liposomal Compound A is administered at 40 mg/kg QOD (Grp 12), or vehicle (Grp 1) using the MDA-MB-436 model.

### Ovarian Cancer

Subcutaneous tumor models were initiated on 6-8 week old, female NOD-scid IL2Rgammanull (NSG) mice. A cell suspension containing 1.5 million OVCAR8 cells in 1:1 PBS:Matrigel was subcutaneously injected into the hind flank of NSG mice. Tumors were measured twice weekly by digital caliper and reported as volume, calculated as V=π×l×w\hat{ }2 wherein l and w denote the longest and shortest tumor diameter respectively. Engrafted mice were then randomized into cohorts for treatment at an average tumor size of 200 mm³.

Orthotopic tumor xenografts were conducted on post-pubescent, 10-13 week old, female NSG mice. 2 million bioluminescent OVCAR8 cells suspended in 500 μL of PBS were inoculated into the intraperitoneal cavity of NSG mice. Diseased mice were then randomized into cohorts and treated at 2-weeks post-inoculation. Tumor burden was assessed by once-weekly in vivo bioluminescent imaging (BLI) and reported as total flux (p/s).

Patient derived xenograft models were established in collaboration with the Princess Margaret Living Biobank. Patient tumor samples were collected, sectioned, and transplanted into the flank of female BALB/c mice. Tumors were then passaged through host BALB/c mice between a minimum of 3-5 passages prior to collection and SC inoculation onto 6-8 week old, female NSG mice. Tumor measurements were taken by digital caliper and reported as volume as described above. Tumors were then grown to an average of 200 mm³ before randomization into evaluated cohorts.

Tables 3-4 for shows the treatment schedules for preliminary dose-finding studies. Table 5 shows the treatment schedule for the efficacy studies (n=6) (i.e., three times per week at every-other-day with a two-day weekend holiday). In all combination studies where both niraparib and ATRi are in a liposomal composition, niraparib and ATRi were in separate liposomal compositions. That is “Lipo NIRB-Comp. A” or “Lipo NIRB-Comp. C” indicates that niraparib and ATRi (Comp. A or Comp. C) are in separate liposomal compositions (i.e., niraparib and ATRi are not co-encapsulated). Efficacy was assessed by both survival and tumor burden. The primary objectives were significant reduction in tumor burden (BLI) and significant improvements in survival. The secondary objective was significant reduction on RBC toxicities.

TABLE 3
Combination of niraparib and Compound A

| | Dose | | | | | |
|---|---|---|---|---|---|---|
| | (mg/kg) | | Dosing | Primary | Secondary | No. of |
| Treatment | NIRB | ATRi | Schedule | Objectives | Objectives | Animals |
| Free NIRB- | 60 | 20 | 3x/weekly; | Significant | Significant | 5 |
| Comp. A | | | 2-4 weeks | reductions in | reduction on | |
| Lipo NIRB- | 60 | 20 | 3x/weekly; | tumor burden | RBC toxicities | 5 |
| Comp. A | | | 2-4 weeks | (BLI) | | |
| (Formulation | | | | Significant | | |
| A) | | | | improvements | | |
| Comp. A Free | | 20 | 3x/weekly; | in survival | | 5 |
| Mono | | | 2-4 weeks | | | |
| Comp. A Lipo | | 20 | 3x/weekly; | | | 5 |
| Mono | | | 2-4 weeks | | | |
| Low PEG | 60 | 20 | 3x/weekly; | | | 5 |
| Lipo NIRB- | | | 2-4 weeks | | | |
| Comp. A | | | | | | |
| (Formulation | | | | | | |
| B) | | | | | | |
| Free NIRB- | 60 | 20 | 3x/weekly; | | | 5 |
| VE822 | | | 2-4 weeks | | | |

| | | | |
|---|---|---|---|
| Vehicle | Matched | 3x/weekly; | 5 |
| | volume | 2-4 weeks | |

| | |
|---|---|
| Total animals | 35 |
| Total animals to order | 40 |
| Volume of NIRB Lipo/Week | 6.0 mL |
| Volume of Comp. A Lipo/Week | 6.0 mL |
| Volume of Low PEG Comp. A Lipo/Week | 3.0 mL |

TABLE 4
Combination of niraparib and Compound C

| | Dose | | | | | |
|---|---|---|---|---|---|---|
| | (mg/kg) | | Dosing | Primary | Secondary | No. of |
| Treatment | NIRB | ATRi | Schedule | Objectives | Objectives | Animals |
| Free NIRB- | 60 | 20 | 3x/weekly; | Significant | Significant | 5 |
| Comp. C | | | 2-4 weeks | reductions in | reduction on | |
| Lipo NIRB- | 60 | 20 | 3x/weekly; | tumor burden | RBC toxicities | 5 |
| Comp. C | | | 2-4 weeks | (BLI) | | |
| (Formulation A) | | | | Significant | | |
| Comp. C Free | | 20 | 3x/weekly; | improvements | | 5 |
| Mono | | | 2-4 weeks | in survival | | |
| Comp. C Lipo | | 20 | 3x/weekly; | | | 5 |
| Mono | | | 2-4 weeks | | | |
| Low PEG Lipo | 60 | 20 | 3x/weekly; | | | 5 |
| NIRB-Comp. C | | | 2-4 weeks | | | |
| (Formulation B) | | | | | | |
| VE822 Mono | 60 | | 3x/weekly; | | | 5 |
| | | | 2-4 weeks | | | |
| NIRB Free | 60 | | 3x/weekly; | | | 5 |
| Mono | | | 2-4 weeks | | | |
| NIRB Lipo | 60 | | 3x/weekly; | | | 5 |
| Mono | | | 2-4 weeks | | | |
| (Formulation A) | | | | | | |

| | | | |
|---|---|---|---|
| Vehicle | Matched | 3x/weekly; | 5 |
| | volume | 2-4weeks | |

| | |
|---|---|
| Total animals | 45 |
| Total animals to order | 57 |
| Volume of NIRB Lipo/Week | 9.0 mL |
| Volume of Comp. C Lipo/Week | 6.0 mL |
| Volume of Low PEG Comp. C Lipo/Week | 3.0 mL |

TABLE 5
Combination of niraparib and Compound A and
anti-cancer agent (cis-platin or irinotecan)

| | Dose (mg/kg) | | | | Dosing |
|---|---|---|---|---|---|
| Treatment | NIRB | ATRi | irinotecan | cisplatin | Schedule |
| Lipo A NIRB- | 60 | 60 | | 1 | 3x/weekly |
| Lipo A Comp. A + | | | | | |
| free cisplatin | | | | | |
| Lipo B NIRB- | 60 | 60 | | 1 | 3x/weekly |
| Lipo B Comp. A + | | | | | |
| free cisplatin | | | | | |
| Lipo A NIRB- | 80 | 80 | | 1 | 3x/weekly |
| Lipo A Comp. A + | | | | | |
| free cisplatin | | | | | |
| Lipo B NIRB- | 80 | 80 | | 1 | 3x/weekly |
| Lipo B Comp. A + | | | | | |
| free cisplatin | | | | | |
| Free irinotecan | | | 50 | | 3x/weekly |
| Lipo B Comp. A | | 80 | | | 3x/weekly |
| Lipo B NIRB | | 80 | | | 3x/weekly |
| Lipo B NIRB + | 80 | 80 | | | 3x/weekly |
| lipo B Comp. A | | | | | |
| Lipo B Comp. A + | | 80 | 50 | | 3x/weekly |
| irinotecan | | | | | |
| Lipo B NIRB + | 80 | | 50 | | 3x/weekly |
| irinotecan | | | | | |
| Lipo B NIRB + | 80 | 80 | 50 | | 3x/weekly |
| Lipo B Comp. A + | | | | | |
| irinotecan | | | | | |

| | | |
|---|---|---|
| Vehicle | Matched volume | 3x/weekly |

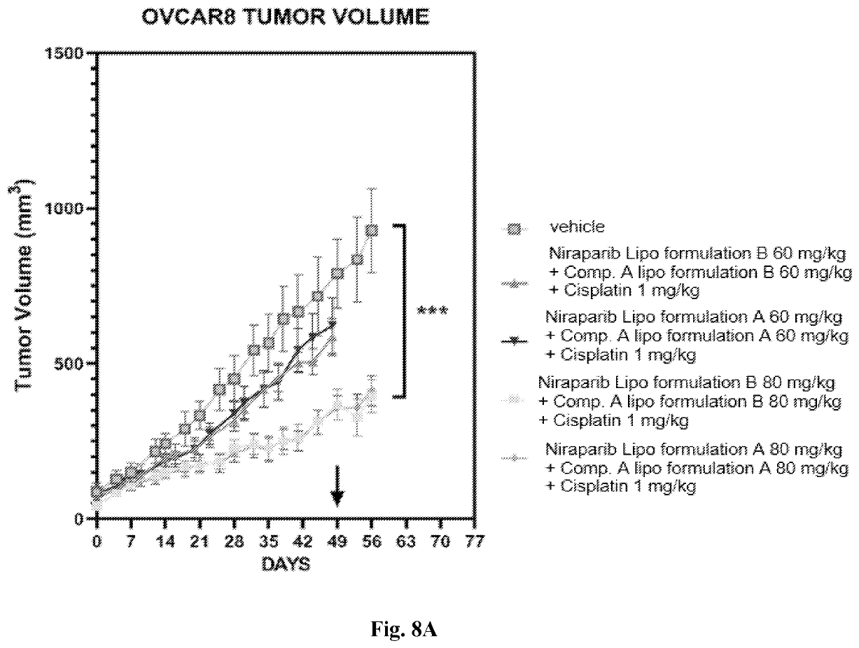

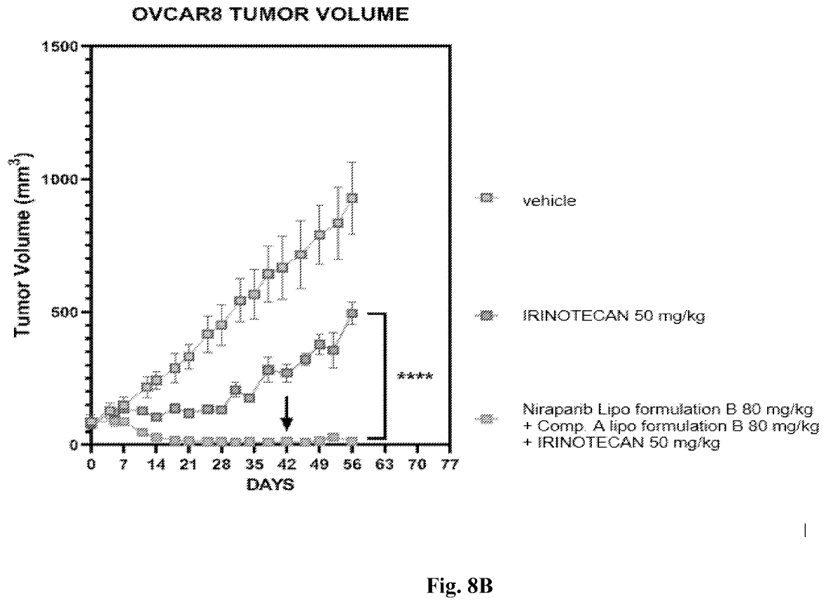

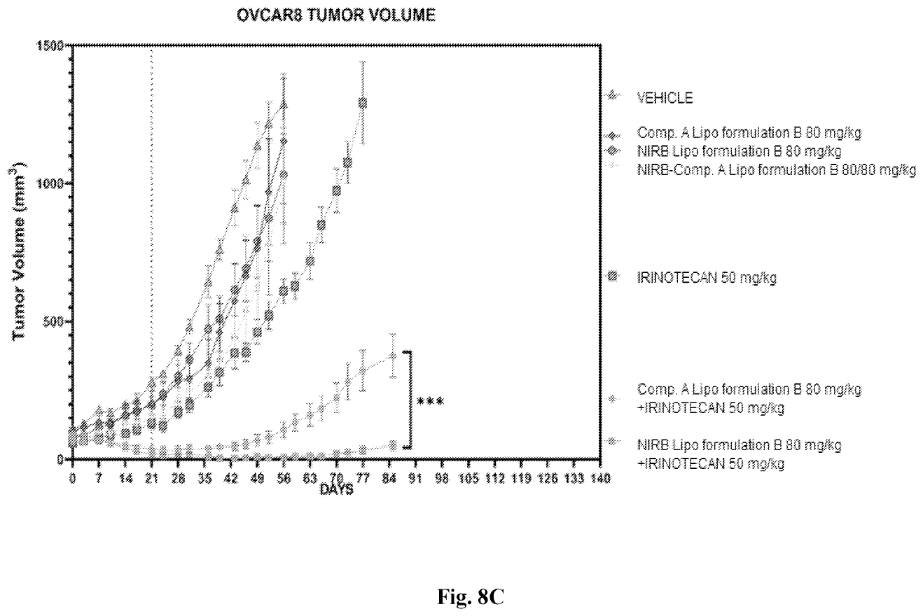

FIG. 8A shows the change in OVCAR8 tumor volume (mm³) after treatment with triple combinations of (i) liposomal niraparib (in Formulation B) administered at 60 mg/kg, liposomal Compound A (in Formulation B) administered at 60 mg/kg, and free cisplatin administered at 1 mg/kg; (ii) liposomal niraparib (in Formulation A) administered at 60 mg/kg, liposomal Compound A (in Formulation A) administered at 60 mg/kg, and free cisplatin administered at 1 mg/kg (iii) liposomal niraparib (in Formulation B) administered at 80 mg/kg, liposomal Compound A (in Formulation B) administered at 80 mg/kg, and free cisplatin administered at 1 mg/kg; and (iv) liposomal niraparib (in Formulation A) administered at 80 mg/kg, liposomal Compound A (in Formulation A) administered at 80 mg/kg, and free cisplatin administered at 1 mg/kg using a OVCAR8 tumor model.

As shown in FIG. 8A the combination of liposomal niraparib administered at 80 mg/kg, and liposomal Compound A administered at 80 mg/kg, resulted in significant reductions in tumor burden compared to vehicle control.

FIG. 8B shows the change in OVCAR8 tumor volume (mm³) after treatment with irinotecan at 50 mg/kg and liposomal niraparib (in Formulation B) administered at 80 mg/kg, liposomal Compound A (in Formulation B) administered at 80 mg/kg, and free irinotecan administered at 50 mg/kg.

FIG. 8C shows the change in OVCAR8 tumor volume (mm³) after treatment with (i) liposomal Compound A (in Formulation B) administered at 80 mg/kg, (ii) liposomal niraparib (in Formulation B) administered at 80 mg/kg, (iii) liposomal Compound A (in Formulation B) administered at 80 mg/kg and liposomal niraparib (in Formulation B) administered at 80 mg/kg; (iv) irinotecan administered sat 50 mg/kg, (v) liposomal Compound A (in Formulation B) administered at 80 mg/kg and irinotecan 50 mg/kg; (vi) liposomal niraparib (in Formulation B) administered at 80 mg/kg and irinotecan administered at 50 mg/kg and (vii) liposomal niraparib (in Formulation B) administered at 80 mg/kg, and liposomal Compound A (in Formulation B) administered at 80 mg/kg and irinotecan administered at 50 mg/kg.

The results of these studies show that triple combination of liposomal niraparib (80 mg/kg) and liposomal Compound A (80 mg/kg) and irinotecan (50 mg/kg) resulted in complete tumor regression that was sustained past the last dose.

### Example 7. Hematology Tolerability Study

In order to study the effects of hematology tolerability under different dosing regimens, 8-12 week old, female C₅₇BL/6 mice were used. Body weight was measured qd×5, then biweekly to the end of the study. Animals were weighed and monitored for survival and in-life observations were collected at the time of weight measurement. Mice were organized into appropriate groups consisting of control (empty vehicle) and treatment groups including free niraparib, free niraparib plus Berzosertib, free niraparib plus Elimusertib and a combination of free niraparib and liposomal Compound A. Mice were given orally, or injected intravenously, with the required volume of sample to administer the prescribed dose to the animals based on individual mouse weights on dosing days. Mice were monitored individually to endpoint which was 18 days. Full volume blood was collected by terminal cardiac puncture under isoflurane anesthesia and analyzed for reticulocyte count/number, red blood cell count/number, hemoglobin, hematocrit, platelet count/number, white blood cell count/number, neutrophil count/number, monocyte count/number and lymphocyte count/number.

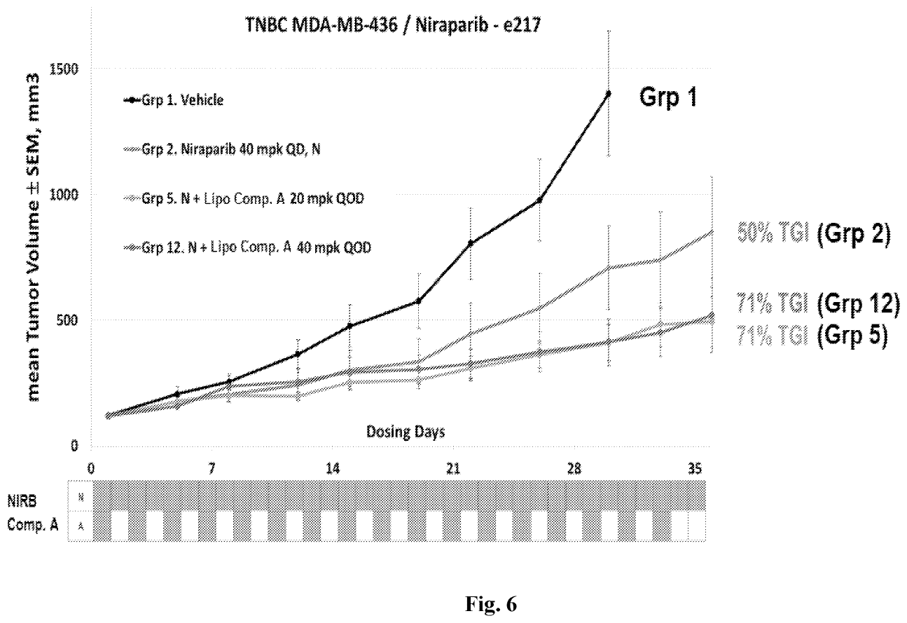

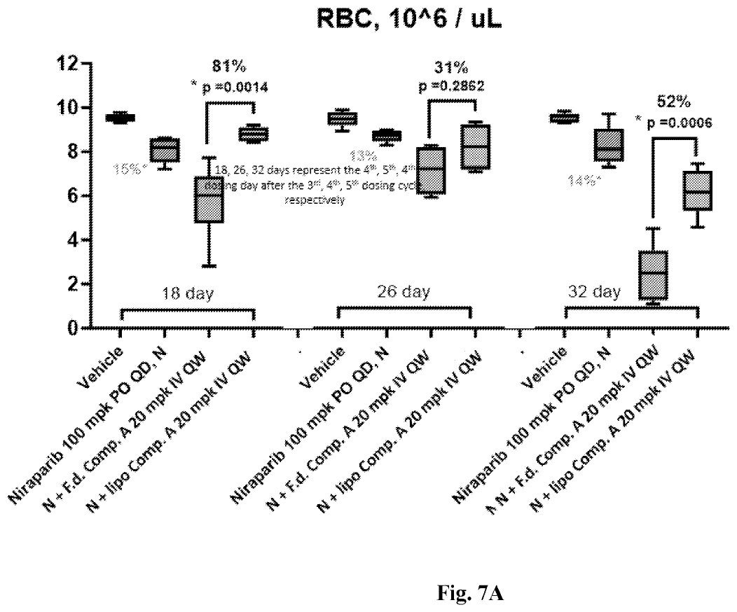

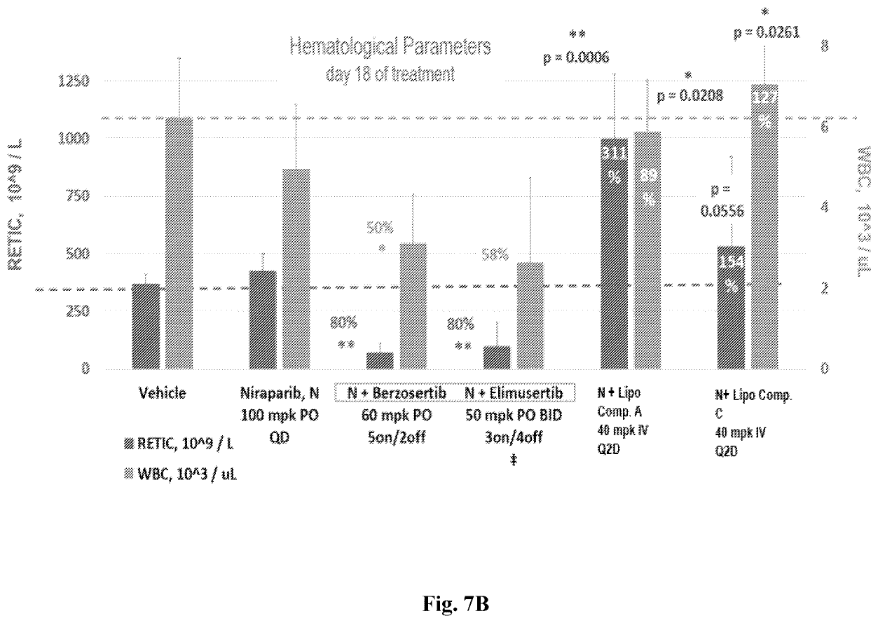

FIG. 7A shows erythroid toxicity of the combination of niraparib and liposomal Compound A vs free drug. Bracketed %'s represent percent improvement of Erythroid toxicity by the liposomal agent vs free drug. These results show that liposomal protection of mature RBC's extended over multiple time points for 5 weeks of niraparib combination treatment.

FIG. 7B shows free niraparib in combination with liposomal Compound A demonstrated complete compensatory recovery of reticulocytopenia (311% improvement over benchmark berzosertib) and attenuation of white blood cell count reduction (89% improvement over berzosertib). FIG. 7B also shows free niraparib in combination with liposomal Compound C demonstrate complete compensatory recovery of reticulocytopenia (154 improvement over benchmark berzosertib) and attenuation of white blood cell count reduction.

Without bound to any theory, these results show that liposomal Compound A can widen the therapeutic index of ATRi/PARPi combinations. Compound A demonstrated an efficacious trend in a BRCA1 mutated TNBC model in combination with a PARPi, niraparib. During the dosing period, the liposomal formulations have comparable activity to the clinical benchmark, with a combined TGI of 71%.

Pan-cytopenia appears to be a key clinical rate-limiting problem in the clinic for ATR inhibitor combinations with both genotoxic chemotherapy and PARP inhibitors. This clinical toxicity was recapitulated in a mouse model using free (liposomal-free) ATR inhibitors, berzosertib and elimusertib, in combination with free niraparib, which induced a severe reticulocytopenia and leukopenia. In contrast, liposomal Compound A rescued these benchmark combination toxicities.

### Example 8. Functional Inhibition Assay

Subcutaneous OVCAR8 tumor models were initiated on 6-8 week old, female NOD-scid IL2Rgammanull (NSG) mice as described in Example 6. At six weeks post inoculation, tumor bearing mice were intravenously given either liposomal niraparib alone, liposomal Compound A alone, liposomal niraparib and liposomal Compound A combination, or vehicle HBS control. Mice (n=3) from each cohort were then intraperitoneally injected with irinotecan at timepoints 24, 48, 72, and 96 hours post compound administration. Mice were then euthanized at 3-hours post irinotecan administration followed by tumor excision. Tumors were then fixed with formalin and paraffin blocked prior to immunohistochemistry staining for the following: γH2AX, pRAD50, pCHK1/2, pKAP1, pATM, RPA32, TUNEL, and H&E. Degree of biomarker activation in tumors of inhibitor treated cohorts was compared with control cohorts (i.e., irinotecan only and treatment naïve mice) using ImageJ analysis.

The publications discussed herein are provided solely for their disclosure prior to the filing date of the present application. Nothing herein is to be construed as an admission that the present invention is not entitled to antedate such publication by virtue of prior invention.

While the invention has been described in connection with proposed specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

### Embodiments

Embodiment 1. A combination comprising:
(i) a compound of formula (I):

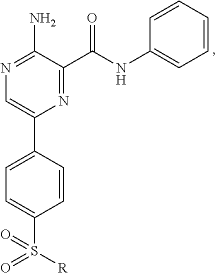

or a pharmaceutically acceptable salt thereof, wherein
R is a moiety comprising an amino group with a pKₐ of greater than 7.0; and
(ii) a PARP inhibitor.

Embodiment 2. The combination of embodiment 1, wherein:
R is —C1-C4 alkylene-NRaRb, -(C1-C4 alkylene)-(heterocyclyl)-(C1-C4 alkyl), -(C1-C4 alkylene)-(heterocyclyl)-(C1-C4 alkylene)-NRaRb, -(C1-C4 alkylene)-(heterocyclyl)-NRaRb, or —NRaRb—(C1-C4 alkyl), wherein the heterocyclyl contains at least one nitrogen as a ring atom; and
Ra and Rb are each independently H or —C1-C4 alkyl.

Embodiment 3. The combination of embodiment 1 or 2, wherein R is —(C₁-C₄ alkylene)-(heterocyclyl)-(C₁-C₄ alkyl), —(C₁-C₄ alkylene)-(heterocyclyl)-(C₁-C₄ alkylene)-NR^{a}R^{b}, or —(C₁-C₄ alkylene)-(heterocyclyl)-NR^{a}R^{b}, wherein the heterocyclyl is a piperidine or a piperazine.

Embodiment 4. The combination of any one of embodiments 1-3, wherein R is:
a)

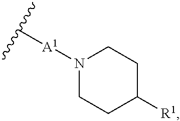

wherein A¹ is absent or C₁-C₄ alkylene, and R¹ is —(C₁-C₄ alkyl)-NR^{a}R^{b}, wherein R^{a} and R^{b} are each independently H or —C₁-C₄ alkyl;
b) —N(R^{a})(C₁-C₄ alkylene)-NR^{a}R^{b}, wherein R^{a} and R^{b} are each independently H or —C₁-C₄ alkyl;
c) —(C₁-C₈ alkyl)-NR^{a}R^{b}; wherein R^{a} and R^{b} are each independently H or C₁-C₄ alkyl; or
d)

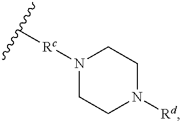

wherein R^{c} is —C₁-C₄ alkylene and R^{d} is —C₁-C₄ alkyl.

Embodiment 5. The combination of any one of embodiments 1-4, wherein the compound of formula (I) is

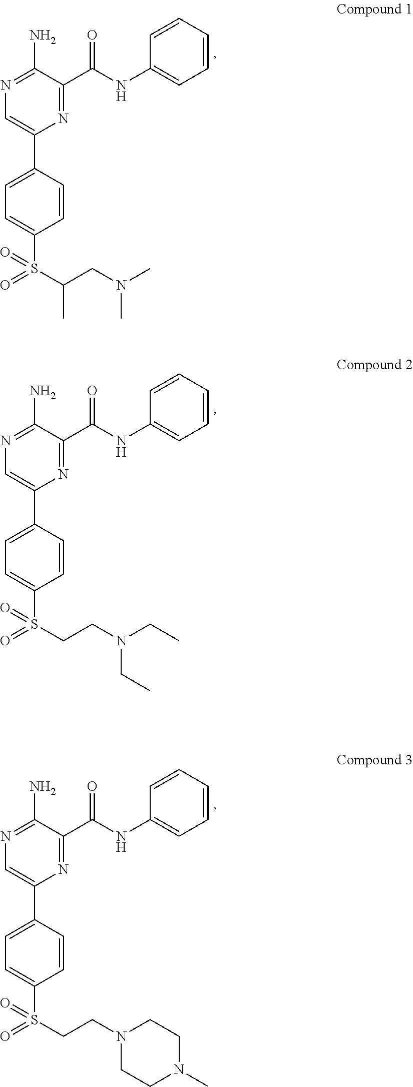 [SMILES: CC(CN(C)C)S(=O)(=O)c1ccc(-c2cnc(N)c(C(=O)Nc3ccccc3)n2)cc1 ; CCN(CC)CCS(=O)(=O)c1ccc(-c2cnc(N)c(C(=O)Nc3ccccc3)n2)cc1 ; CN1CCN(CCS(=O)(=O)c2ccc(-c3cnc(N)c(C(=O)Nc4ccccc4)n3)cc2)CC1]

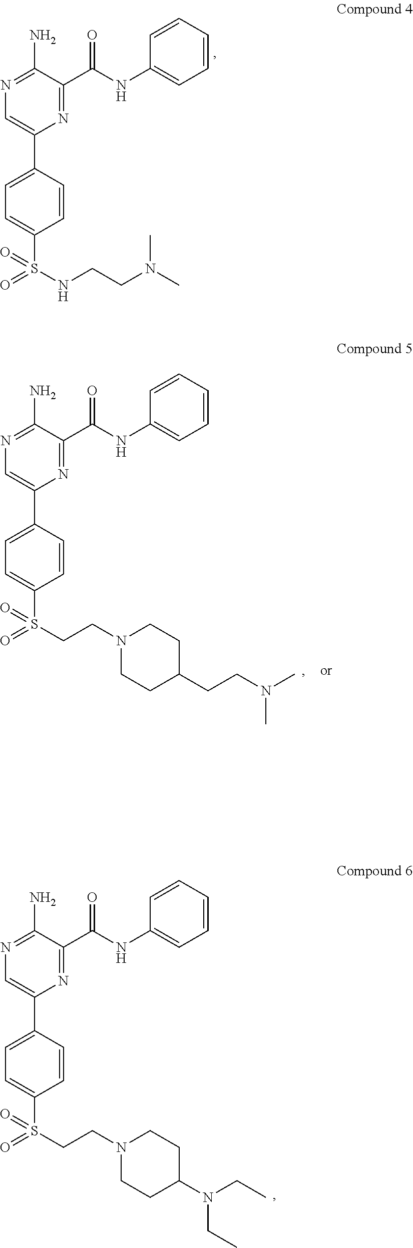 [SMILES: CN(C)CCNS(=O)(=O)c1ccc(-c2cnc(N)c(C(=O)Nc3ccccc3)n2)cc1 ; CN(C)CCC1CCN(CCS(=O)(=O)c2ccc(-c3cnc(N)c(C(=O)Nc4ccccc4)n3)cc2)CC1 ; CCN(CC)C1CCN(CCS(=O)(=O)c2ccc(-c3cnc(N)c(C(=O)Nc4ccccc4)n3)cc2)CC1]

or a pharmaceutically acceptable salt thereof.

Embodiment 6. The combination of any one of embodiments 1-5, wherein the PARP inhibitor is olaparib, rucaparib, niraparib, talazoparib, veliparib, fluzoparib, pamiparib, fluazolepali, amelparib, simmiparib, mefuparib, iniparib, stenoparib, senaparib, atamparib, venadaparib, or nesuparib, or a pharmaceutically acceptable salt thereof.

Embodiment 7. The combination of any one of embodiments 1-6, wherein the PARP inhibitor is niraparib.

Embodiment 8. The combination of any one of embodiments 1-7, wherein the combination comprises a pharmaceutical composition comprising the compound of formula (I) or a pharmaceutically acceptable salt thereof.

Embodiment 9. The combination of any one of embodiments 1-8, wherein the combination comprises a pharmaceutical composition comprising the PARP inhibitor.

Embodiment 10. The combination of embodiment 8 or 9, wherein the pharmaceutical composition comprising the compound of formula (I) or a pharmaceutically acceptable salt thereof and the pharmaceutical composition comprising the PARP inhibitor are different pharmaceutical compositions.

Embodiment 11. The combination of embodiment 8 or 9, wherein the pharmaceutical composition comprising the compound of formula (I) or a pharmaceutically acceptable salt thereof and the pharmaceutical composition comprising the PARP inhibitor are the same pharmaceutical composition.

Embodiment 12. The combination of any one of embodiments 8-11, wherein the pharmaceutical composition comprising the compound of formula (I) or a pharmaceutically acceptable salt thereof is a liposomal composition.

Embodiment 13. The combination of any one of embodiments 8-11, wherein the pharmaceutical composition comprising the PARP inhibitor is a liposomal composition.

Embodiment 14. The combination of embodiment 12 or 13, wherein liposome of the liposomal composition comprises one or more phospholipids.

Embodiment 15. The combination of embodiment 14, wherein the phospholipid is 1,2-distearoyl-sn-glycero-3-phosphocholine (DSPC).

Embodiment 16. The combination of any one of embodiments 12-15, wherein liposome of the liposomal composition comprises cholesterol.

Embodiment 17. The combination of any one of embodiments 12-16, wherein liposome of the liposomal composition comprises PEG(2000)-distearoylglycerol (PEG-DSG).

Embodiment 18. The combination of any one of embodiments 12-17, wherein liposome of the liposomal composition comprises DSPC and cholesterol.

Embodiment 19. The combination of any one of embodiments 12-18, wherein liposome of the liposomal composition comprises DSPC, cholesterol, and PEG-DSG.

Embodiment 20. The combination of any one of embodiments 15, 18, and 19, wherein the liposome comprises about 50 mol % to about 70 mol % of DSPC.

Embodiment 21. The combination of embodiment 20, wherein the liposome comprises about 55 mol %, about 56 mol %, about 57 mol %, about 58 mol %, about 59 mol %, about 60 mol %, about 61 mol %, about 62 mol %, about 63 mol %, about 64 mol %, or about 65 mol % of DSPC.

Embodiment 22. The combination of any one of embodiments 16, 18 and 19, wherein the liposome comprises about 30 mol % to about 50 mol % of cholesterol.

Embodiment 23. The combination of embodiment 22, wherein the liposome comprises about 35 mol %, about 36 mol %, about 37 mol %, about 38 mol %, about 39 mol %, about 40 mol %, about 41 mol %, about 42 mol %, about 43 mol %, about 44 mol %, or about 45 mol % of cholesterol.

Embodiment 24. The combination of any one of embodiments 17-19, wherein the liposome comprises about 0.1 mol % to about 5 mol % of PEG-DSG.

Embodiment 25. The combination of embodiment 24, wherein the liposome comprises about 0.1 mol %, about 0.15 mol %, about 0.2 mol %, about 0.25 mol %, about 0.3 mol %, about 0.35 mol %, about 0.4 mol %, about 0.45 mol %, about 0.5 mol %, about 0.6 mol %, about 0.7 mol %, about 0.8 mol %, about 0.9 mol %, about 1.0 mol %, about 1.5 mol %, about 2.0 mol %, about 2.5 mol %, about 3.0 mol %, about 3.5 mol %, about 4.0 mol %, about 4.5 mol %, or about 5.0 mol % of PEG-DSG.

Embodiment 26. The combination of any one of embodiments 1-24, wherein the combination further comprises another pharmaceutically active agent.

Embodiment 27. A method for treating cancer, comprising administering to a subject in need thereof an effective amount of the combination any one of embodiments 1-26.

Embodiment 28. The method of embodiment 27, wherein the combination comprises a pharmaceutical composition comprising the compound of formula (I) or a pharmaceutically acceptable salt thereof.

Embodiment 29. The method of embodiment 27 or 28, wherein the combination comprises a pharmaceutical composition comprising the PARP inhibitor.

Embodiment 30. The method of embodiment 28 or 29, wherein the pharmaceutical composition comprising the compound of formula (I) or a pharmaceutically acceptable salt thereof and the pharmaceutical composition comprising the PARP inhibitor are different pharmaceutical compositions.

Embodiment 31. The method of embodiment 28 or 29, wherein the pharmaceutical composition comprising the compound of formula (I) or a pharmaceutically acceptable salt thereof and the pharmaceutical composition comprising the PARP inhibitor are the same pharmaceutical composition.

Embodiment 32. The method of any one of embodiments 28-31, wherein the pharmaceutical composition comprising the compound of formula (I) or a pharmaceutically acceptable salt thereof is a liposomal composition.

Embodiment 33. The method of any one of embodiments 28-31, wherein the pharmaceutical composition comprising the PARP inhibitor is a liposomal composition.

Embodiment 34. The method of any one of embodiments 28-33, wherein the compound of formula (I) or a pharmaceutically acceptable salt thereof and the PARP inhibitor provide therapeutic synergy.

Embodiment 35. The method of any one of embodiments 28-34, wherein the cancer is breast cancer, ovarian cancer, pancreatic cancer, prostate cancer, colorectal cancer, lung cancer, brain cancer, melanoma, or osteosarcoma.

Embodiment 36. The method of embodiment 35, wherein the breast cancer is triple negative breast cancer.

Embodiment 37. The method of embodiment 35, wherein the lung cancer is small cell lung cancer or non-small cell lung cancer.

Embodiment 38. The method of any one of embodiments 33-37, wherein the combination does not cause acute neurological toxicity.

Embodiment 39. The method of any one of embodiments 33-37, wherein the combination does not cause long-term hematological toxicity.

What is claimed is:

1. A combination comprising:
(i) a compound of formula (I): 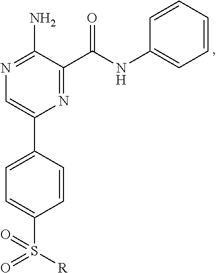
or a pharmaceutically acceptable salt thereof, wherein
R is a moiety comprising an amino group with a pKₐ of greater than 7.0; and
(ii) a PARP inhibitor.

2. The combination of claim 1, wherein:
R is —C₁-C₄ alkylene-NR^{a}R^{b}, —(C₁-C₄ alkylene)-(heterocyclyl)-(C₁-C₄ alkyl), —(C₁-C₄ alkylene)-(heterocyclyl)-(C₁-C₄ alkylene)-NR^{a}R^{b}, —(C₁-C₄ alkylene)-(heterocyclyl)-NR^{a}R^{b}, or —NR^{a}R^{b}—(C₁-C₄ alkyl), wherein the heterocyclyl contains at least one nitrogen as a ring atom; and
R^{a} and R^{b} are each independently H or —C₁-C₄ alkyl.

3. The combination of claim 1 or 2, wherein R is —(C₁-C₄ alkylene)-(heterocyclyl)-(C₁-C₄ alkyl), —(C₁-C₄ alkylene)-(heterocyclyl)-(C₁-C₄ alkylene)-NR^{a}R^{b}, or —(C₁-C₄ alkylene)-(heterocyclyl)-NR^{a}R^{b}, wherein the heterocyclyl is a piperidine or a piperazine.

4. The combination of any one of claims 1-3, wherein R is:
a) 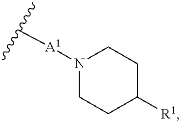
wherein A¹ is absent or C₁-C₄ alkylene, and R¹ is —(C₁-C₄ alkyl)-NR^{a}R^{b}, wherein R^{a} and R^{b} are each independently H or —C₁-C₄ alkyl;
b) —N(R^{a})(C₁-C₄ alkylene)-NR^{a}R^{b}, wherein R^{a} and R^{b} are each independently H or —C₁-C₄ alkyl;
c) —(C₁-C₈ alkyl)-NR^{a}R^{b}, wherein R^{a} and R^{b} are each independently H or C₁-C₄ alkyl; or
d) 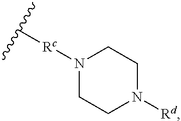
wherein R^{c} is —C₁-C₄ alkylene and R^{d} is —C₁-C₄ alkyl.

5. The combination of any one of claims 1-4, wherein the compound of formula (I) is 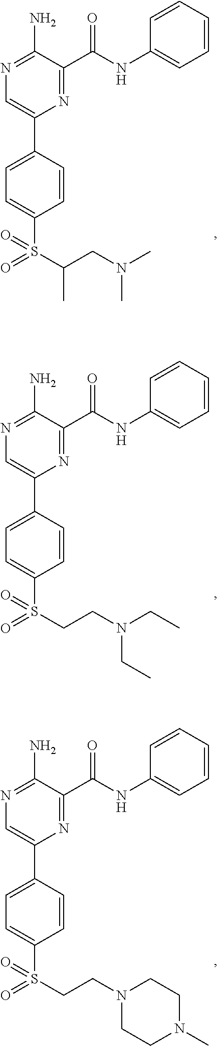 [SMILES: CC(CN(C)C)S(=O)(=O)c1ccc(-c2cnc(N)c(C(=O)Nc3ccccc3)n2)cc1 ; CCN(CC)CCS(=O)(=O)c1ccc(-c2cnc(N)c(C(=O)Nc3ccccc3)n2)cc1 ; CN1CCN(CCS(=O)(=O)c2ccc(-c3cnc(N)c(C(=O)Nc4ccccc4)n3)cc2)CC1] 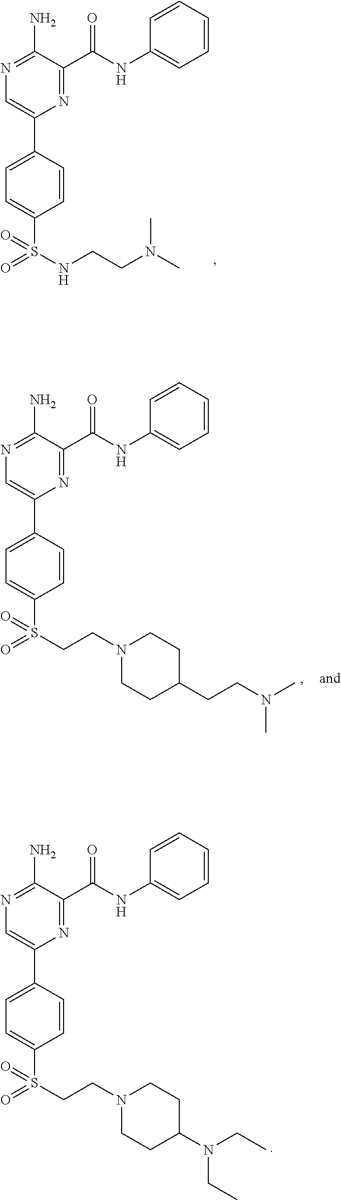 [SMILES: CN(C)CCNS(=O)(=O)c1ccc(-c2cnc(N)c(C(=O)Nc3ccccc3)n2)cc1 ; CN(C)CCC1CCN(CCS(=O)(=O)c2ccc(-c3cnc(N)c(C(=O)Nc4ccccc4)n3)cc2)CC1 ; CCN(CC)C1CCN(CCS(=O)(=O)c2ccc(-c3cnc(N)c(C(=O)Nc4ccccc4)n3)cc2)CC1]
or a pharmaceutically acceptable salt thereof.

6. The combination of any one of claims 1-5, wherein the PARP inhibitor is olaparib, rucaparib, niraparib, talazoparib, veliparib, fluzoparib, pamiparib, fluazolepali, amelparib, simmiparib, mefuparib, iniparib, stenoparib, senaparib, atamparib, venadaparib, or nesuparib, or a pharmaceutically acceptable salt thereof.

7. The combination of any one of claims 1-6, wherein the PARP inhibitor is niraparib.

8. The combination of any one of claims 1-7, wherein the combination comprises a pharmaceutical composition comprising the compound of formula (I) or a pharmaceutically acceptable salt thereof.

9. The combination of any one of claims 1-8, wherein the combination comprises a pharmaceutical composition comprising the PARP inhibitor.

10. The combination of claim 8 or 9, wherein the pharmaceutical composition comprising the compound of formula (I) or a pharmaceutically acceptable salt thereof and the pharmaceutical composition comprising the PARP inhibitor are different pharmaceutical compositions.

11. The combination of claim 8 or 9, wherein the pharmaceutical composition comprising the compound of formula (I) or a pharmaceutically acceptable salt thereof and the pharmaceutical composition comprising the PARP inhibitor are the same pharmaceutical composition.

12. The combination of any one of claims 8-11, wherein the pharmaceutical composition comprising the compound of formula (I) or a pharmaceutically acceptable salt thereof is a liposomal composition.

13. The combination of any one of claims 8-11, wherein the pharmaceutical composition comprising the PARP inhibitor is a liposomal composition.

14. The combination of claim 12 or 13, wherein liposome of the liposomal composition comprises one or more phospholipids.

15. The combination of claim 14, wherein the phospholipid is 1,2-distearoyl-sn-glycero-3-phosphocholine (DSPC).

16. The combination of any one of claims 12-15, wherein liposome of the liposomal composition comprises cholesterol.

17. The combination of any one of claims 12-16, wherein liposome of the liposomal composition comprises PEG(2000)-distearoylglycerol (PEG-DSG).

18. The combination of any one of claims 12-17, wherein liposome of the liposomal composition comprises DSPC and cholesterol.

19. The combination of any one of claims 12-18, wherein liposome of the liposomal composition comprises DSPC, cholesterol, and PEG-DSG.

20. The combination of any one of claims 15, 18, and 19, wherein the liposome comprises about 50 mol % to about 70 mol % of DSPC.

21. The combination of claim 20, wherein the liposome comprises about 55 mol %, about 56 mol %, about 57 mol %, about 58 mol %, about 59 mol %, about 60 mol %, about 61 mol %, about 62 mol %, about 63 mol %, about 64 mol %, or about 65 mol % of DSPC.

22. The combination of any one of claims 16, 18 and 19, wherein the liposome comprises about 30 mol % to about 50 mol % of cholesterol.

23. The combination of claim 22, wherein the liposome comprises about 35 mol %, about 36 mol %, about 37 mol %, about 38 mol %, about 39 mol %, about 40 mol %, about 41 mol %, about 42 mol %, about 43 mol %, about 44 mol %, or about 45 mol % of cholesterol.

24. The combination of any one of claims 17-19, wherein the liposome comprises about 0.1 mol % to about 5 mol % of PEG-DSG.

25. The combination of claim 24, wherein the liposome comprises about 0.1 mol %, about 0.15 mol %, about 0.2 mol %, about 0.25 mol %, about 0.3 mol %, about 0.35 mol %, about 0.4 mol %, about 0.45 mol %, about 0.5 mol %, about 0.6 mol %, about 0.7 mol %, about 0.8 mol %, about 0.9 mol %, about 1.0 mol %, about 1.5 mol %, about 2.0 mol %, about 2.5 mol %, about 3.0 mol %, about 3.5 mol %, about 4.0 mol %, about 4.5 mol %, or about 5.0 mol % of PEG-DSG.

26. The combination of any one of claims 1-24, wherein the combination further comprises another pharmaceutically active agent.

27. The combination of claim 26, wherein the combination further comprises an anti-cancer agent.

28. The combination of claim 27, wherein the anti-cancer agent is cisplatin.

29. The combination of claim 27, wherein the anti-cancer agent is irinotecan or a pharmaceutically acceptable salt thereof.

30. A combination comprising:
(i) a PARP inhibitor in a liposomal composition; and
(ii) an anti-cancer agent.

31. The combination of claim 30, wherein liposome of the liposomal composition comprises one or more phospholipids.

32. The combination of claim 31, wherein the phospholipid is 1,2-distearoyl-sn-glycero-3-phosphocholine (DSPC).

33. The combination of any one of claims 30-32, wherein liposome of the liposomal composition comprises cholesterol.

34. The combination of any one of claims 30-33, wherein liposome of the liposomal composition comprises PEG(2000)-distearoylglycerol (PEG-DSG).

35. The combination of any one of claims 30-34, wherein liposome of the liposomal composition comprises DSPC and cholesterol.

36. The combination of any one of claims 30-35, wherein liposome of the liposomal composition comprises DSPC, cholesterol, and PEG-DSG.

37. The combination of any one of claims 32, 35, and 36, wherein the liposome comprises about 50 mol % to about 70 mol % of DSPC.

38. The combination of claim 37, wherein the liposome comprises about 55 mol %, about 56 mol %, about 57 mol %, about 58 mol %, about 59 mol %, about 60 mol %, about 61 mol %, about 62 mol %, about 63 mol %, about 64 mol %, or about 65 mol % of DSPC.

39. The combination of any one of claims 33, 35, and 36, wherein the liposome comprises about 30 mol % to about 50 mol % of cholesterol.

40. The combination of claim 39, wherein the liposome comprises about 35 mol %, about 36 mol %, about 37 mol %, about 38 mol %, about 39 mol %, about 40 mol %, about 41 mol %, about 42 mol %, about 43 mol %, about 44 mol %, or about 45 mol % of cholesterol.

41. The combination of any one of claims 34-36, wherein the liposome comprises about 0.1 mol % to about 5 mol % of PEG-DSG.

42. The combination of claim 41, wherein the liposome comprises about 0.1 mol %, about 0.15 mol %, about 0.2 mol %, about 0.25 mol %, about 0.3 mol %, about 0.35 mol %, about 0.4 mol %, about 0.45 mol %, about 0.5 mol %, about 0.6 mol %, about 0.7 mol %, about 0.8 mol %, about 0.9 mol %, about 1.0 mol %, about 1.5 mol %, about 2.0 mol %, about 2.5 mol %, about 3.0 mol %, about 3.5 mol %, about 4.0 mol %, about 4.5 mol %, or about 5.0 mol % of PEG-DSG.

43. The combination of any one of claims 30-42, wherein the anti-cancer agent is cisplatin.

44. The combination of any one of claims 30-42, wherein the anti-cancer agent is irinotecan or a pharmaceutically acceptable salt thereof.

45. The combination of any one of claims 30-44, wherein the PARP inhibitor is olaparib, rucaparib, niraparib, talazoparib, veliparib, fluzoparib, pamiparib, fluazolepali, amelparib, simmiparib, mefuparib, iniparib, stenoparib, senaparib, atamparib, venadaparib, or nesuparib, or a pharmaceutically acceptable salt thereof.

46. The combination of any one of claims 30-45, wherein the PARP inhibitor is niraparib.

47. A method for treating cancer, comprising administering to a subject in need thereof an effective amount of the combination any one of claims 1-46.

48. The method of claim 47, wherein the combination provides therapeutic synergy.

49. The method of claim 47 or 48, wherein the cancer is breast cancer, ovarian cancer, pancreatic cancer, prostate cancer, colorectal cancer, lung cancer, brain cancer, melanoma, or osteosarcoma.

50. The method of claim 49, wherein the breast cancer is triple negative breast cancer.

51. The method of claim 49, wherein the lung cancer is small cell lung cancer or non-small cell lung cancer.

52. The method of any one of claims 47-50, wherein the combination does not cause acute neurological toxicity.

53. The method of any one of claims 47-50, wherein the combination does not cause long-term hematological toxicity.
